(19) United States
(12) Patent Application Publication
(10) Pub. No.: US 2011/0250454 A1
(43) Pub. Date: Oct. 13, 2011

(54) PREPARATION OF NEBIVOLOL

(76) Inventors: Pranab Haldar, Midnapur (West) (IN); China Malakondaiah Golla, Hyderabad (IN); Akula Swapna, Hyderabad (IN); Karri Vijaya Kumar, East Godavari (IN); Suhas Jawlekar, Parbhani (IN); Arjun Kumar Tummala, Visakhapatnam (IN); Deepali Patro, Hyderabad (IN); Sesha Reddy Yarraguntla, Hyderabad (IN); Srividya Ramakrishnan, Hyderabad (IN); Venkata Ramana Reddy Chimala, Hyderabad (IN)

(21) Appl. No.: 13/082,439

(22) Filed: Apr. 8, 2011

Related U.S. Application Data
(60) Provisional application No. 61/348,370, filed on May 26, 2010.
(60) Provisional application No. 61/367,942, filed on Jul. 27, 2010.
(60) Provisional application No. 61/427,953, filed on Dec. 29, 2010.

(30) Foreign Application Priority Data
Apr. 9, 2010 (IN) 983/CHE/2010
Jun. 3, 2010 (IN) 1531/CHE/2010
Nov. 11, 2010 (IN) 3376/CHE/2010

(51) Int. Cl. C07D 407/12

(52) U.S. Cl. 428/402; 549/407

(57) ABSTRACT

Processes for the synthesis of pharmacologically active 2,2-iminobisethanol derivatives, e.g., 2H-1-benzopyran-2 methanol-α,α′-iminobis(methylene)]bis-[6-fluoro-3,4-dihydro-[2R*[R*[R*(S*)]]]], and their pharmaceutically acceptable salts.

Aspects of the present application relate to processes for synthesizing pharmacologically active 2,2-iminobisethanol derivatives, e.g., 2H-1-benzopyran-2 methanol-α,α′-iminobis(methylene)]bis-[6-fluoro-3,4-dihydro-[2R*[R*[R*(S*)]]]], and their pharmaceutically acceptable salts.

The drug compound having the adopted name “nebivolol” is a highly selective β₁ blocker and has been found to be useful for the management of hypertension. Chemical names for nebivolol are: [2R*[R*[R*S*)]-α,α′-(iminobismethylene)bis[6-fluoro-2-chromanmethanol]; and (1RS,1′RS)-1,1′-[(2RS,2′SR)-bis(6-fluoro-3,4-dihydro-2H-1-benzopyran-2-yl)]-2,2′-iminodiethanol.

Nebivolol, in the form of its hydrochloride salt, is the active ingredient in products sold as BYSTOLIC®, and the product prescribing information discloses nebivolol to be a mixture of equal amounts of two enantiomers, having respectively the SRRR- and the RSSS-configurations. The SRRR configuration is called d-nebivolol and the RSSS configuration is called l-nebivolol.

Methods for preparation of nebivolol and its intermediate compounds are disclosed in European Patents 0145067 and 0334429. European Patent 0334429 B1 discloses a process for the preparation of specifically the RSSS isomer of nebivolol. The process for preparing RSSS nebivolol involves the use of hazardous reagents like thionyl chloride, sodium hydride, and diisobutyl aluminium hydride (DIBAL), expensive optically active reagents like (+)-1,2,3,4,4a,9,10,10a-octahydro-1,4a-dimethyl-7-(1-methylethyl-1-phenathrenemethanamine [(+)-dehydroabietylamine], and uses combersome column chromatography and low temperatures. The processes also involve a large number of steps, thereby increasing the energy, manpower, and time required to complete a production cycle, rendering the processes commercially disadvantageous.

European Patent 744946 B1 describes a process for obtaining nebivolol hydrochloride from a mixture containing the desired (RSSS+SRRR) nebivolol base, contaminated with the undesired (RSRR+SRSS) diastereomers, using ethanol as both a reactant and the recrystallization solvent. A major disadvantage of this process is a very low yield (6.6%) of the desired isomers (having the SRRR- and the RSSS-configuration) of nebivolol hydrochloride. Moreover, ethanol is a solvent that can be used only in controlled quantities due to regulatory requirements, and therefore its usefulness on an industrial scale is limited.

International Application Publication Nos. WO 2006/025070 A2, WO 2006/016376 A1, WO 2007/083318 A1, and WO 2009/082913 A1, and India Patent Application 2008/CH/01642 are directed to processes for preparing nebivolol and its hydrochloride salt. In the processes of these applications, N-protected nebivolol has been subjected to purification, either by isolation at low temperatures, by fractional crystallization of acid addition salts of N-protected nebivolol, or by fractional crystallization of N-protected nebivolol free base, using ether as a solvent, or by purification using a solvent-antisolvent technique.

International Application Publication Nos. WO 2010/049455 A1 and WO 2010/089764 A2 are related to processes for preparation of nebivolol, and also disclose substantially pure nebivolol hydrochloride in view of the “de-mono-F” nebivolol and other process-related impurities.

European Patent Application 2163551 A1 and International Application Publication WO 2010/049455 A1 relate to processes for preparing nebivolol, by deprotection of N-benzylated nebivolol under modified conditions.

Stereoselective methods for the preparation of l-nebivolol and d-nebivolol, and their intermediate compounds, are described in International Application Publication Nos. WO 2009/121710 A2, WO 2008/064827 A2, WO 2008/064826 A1, WO 2008/040528 A2, and W02010034927 A1.

In most of the aforementioned patents or applications, N-benzylated nebivolol in racemic form, and/or its pure enantiomers, are deprotected by catalytic hydrogenation procedures such as using palladium or platinum supported on carbon in a suitable solvent. Basically, the benzyl group is removed by a classic hydrogenation in the presence of 10% Pd on C as a catalyst.

Among the aforementioned patents or applications, in International Application Publication Nos. WO 2008/064827 A2 and WO 2010/049455 A1, debenzylation is accomplished by catalytic hydrogen transfer reductions (CTH) wherein a source of molecular hydrogen is either ammonium formate or formic acid, in the presence of 5% or 10% of Pd on carbon.

The WO 2010/049455 publication mentions that deprotection by catalytic transfer hydrogenation exhibits the drawback of being slow, and of not leading to complete conversions due to progressive poisoning of the catalyst by the amines which are generated as products of the N-debenzylation reaction. The publication also teaches that conventional hydrogenation processes lead to unwanted dehalogenated compounds in large amounts (>1%) and the necessary subsequent purifications by re-crystallizations of the end product, besides being expensive in terms of time, cost and consumer materials, fail to limit the de-fluorinated impurity below 0.1% required by pharmaceutical standards. According to the publication, catalytic transfer hydrogenation in presence of formic acid with 10% of Pd on C is chemoselective and limits the formation of an undesired dehalogenated by-product, i.e., “de-mono-F” nebivolol having the formula below.

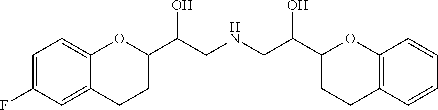

The methods described above either involve a multiplicity of steps, give low yields, or do not result in desired isomeric purity of the final compound. In view of demerits associated with prior art processes there remains a need for methods for the synthesis of substantially pure nebivolol and its salts that eliminate or reduce in terms of quantity the use of hazardous chemicals like DIBAL, thionyl chloride and heterogeneous catalyst like palladium on charcoal, while at the same time reducing the number of processing steps and controlling the undesired dehalogenated by-product, i.e., “de-mono-F” nebivolol.

In most of the synthetic routes previously disclosed, the compound 6-fluoro-3,4-dihydro-2H-1-benzopyran-2-carboxaldehyde is an intermediate employed for the preparation of nebivolol or its hydrochloride salt. In the synthetic route according to European Patent 0145067, the aldehyde intermediate is obtained from an acid intermediate by a sequence of reactions. First, the acid intermediate is esterified with ethanol under acidic conditions, to provide the corresponding ethyl 6-fluoro-3,4-dihydro-2H-1-benzopyran-2-carboxylate. The ethyl ester is then reduced to the corresponding alcohol, using VITRIDE® under a nitrogen atmosphere, to afford 6-fluoro-3,4-dihydro-2H-1-benzopyran-2-methanol, which is subsequently oxidized to the corresponding aldehyde by means of Swern oxidation using oxalyl chloride, and dimethylsulfoxide and dichloromethane as a solvent, at −60° C. under a nitrogen atmosphere.

In the synthetic route according to European Patent 0334429, the aldehyde intermediate is obtained from the acid intermediate, by first esterifying in methanol using sulfuric acid under reflux conditions, to afford a methyl ester as an oily residue, and subsequently reducing to the corresponding aldehyde using DIBAL at a temperature of −80° C. under a nitrogen atmosphere.

India Patent No. 221733 discloses another process for preparation of the aldehyde intermediate, where the acid intermediate is first converted to a carboxylic acid amide in the presence of an acid activating agent, followed by reduction of the amide with an alkoxy metallic hydride.

Additionally, Indian Patent Application 787/KOL/2008 describes a process for preparation of 6-fluoro-3,4-dihydro-2H-benzopyran-2-carboxaldehyde, comprising esterification of 6-fluoro-3,4-dihydro-2H-1-benzopyran-2-carboxylic acid to obtain an ester, reduction of the ester to afford 6-fluoro-3,4-dihydro-2H-1-benzopyran-2-methanol, and oxidation of the alcohol with an alkali metal hypochlorite in the presence of tetramethylpiperidine-1-oxyl (TEMPO) and an alkali metal halide, to obtain 6-fluoro-3,4-dihydro-2H-benzopyran-2-carboxaldehyde.

The solid state physical properties of an active pharmaceutical ingredient (API), such as nebivolol hydrochloride, can be very important in formulating pharmaceutical products, having sometimes profound effects on the ease of formulating and reproducibility of formulations. Particle sizes, for example, may affect the flowability and mixability of a drug substance. Additionally, pharmaceutical stability is believed to depend on simultaneous influence of various factors, of which some important factors include the sizes of crystals, shapes of crystals, water content, residual solvents, and impurities.

The literature reveals that attempts to use the natural crystalline form of nebivolol hydrochloride have resulted in poor dissolution rate and poor bioavailability. Attempts for combining the crystalline form with a wetting agent are largely unsuccessful. For achieving appropriate dissolution rate or bioavailability of nebivolol hydrochloride, micronization was required. U.S. Pat. No. 5,759,580 discloses a micronized form of nebivolol hydrochloride having a specific surface area of at least 23×10³ cm²/g, which is employed for preparation of its pharmaceutical composition in the presence of a wetting agent.

International Application Publication No. WO 2006/025070 A2 discusses disadvantages of micronization and use of wetting agents for formulation, and further discloses a pharmaceutical composition wherein crystalline nebivolol hydrochloride is used as is for formulation processes; thus, micronization is not employed. The application does not disclose the particle size or surface area of nebivolol hydrochloride employed for preparation of its pharmaceutical composition. Though it is mentioned that the preferred specific surface area of the nebivolol hydrochloride is 0.2×10³ m²/Kg to 1.95×10³ m²/Kg, there is no disclosure about whether this specific surface area is achieved directly from the reaction, or some other process was employed to achieve the desired result.

There are disadvantages associated with micronization, and specific surface areas are important for obtaining an appropriate dissolution rate or bioavailability of nebivolol hydrochloride in formulations.

The methods described above either involve multiple steps, give low yields, or do not result in desired purities of the final compound. There remains a need for methods for the synthesis of nebivolol and its salts that eliminate the use of hazardous chemicals like DIBAL and thionyl chloride, while at the same time reducing the number of steps in the process.

### SUMMARY

Aspects of the present application provide processes for the preparation of nebivolol acid salts of Formula I, and its intermediate compounds. Such salts can be converted to nebivolol, if desired, using techniques that are well-known to those having skill in the art. Each step of the processes disclosed herein is contemplated both in the context of the multi-step sequences described, and individually.

A general preparation of nebivolol acid salts according to embodiments of the present application proceeds as shown in the reaction scheme of FIG. 5, where P is a protecting group and HX is an acid.

In an aspect, there are provided processes for the preparation of nebivolol acid salts of Formula I, embodiments comprising at least one of the steps:

a) Reacting a compound of Formulas III or III′ with an acid in an organic solvent, to afford the compound of Formula II or II′.

b) Purifying a compound of Formulas II or II′ by suitable purification techniques in an organic solvent, aqueous organic solvent, or mixtures thereof, to afford the compound of Formula I, and optionally converting the compound of Formula I to a hydrochloride salt, if HX in Formula I is not HCl.

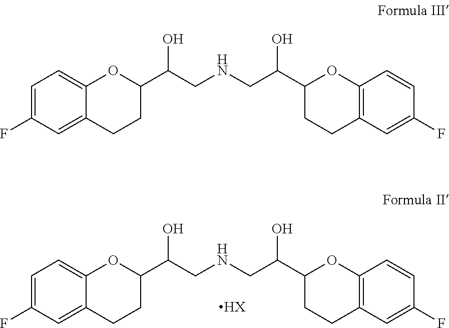

According to an aspect of the application, there are provided processes for preparing the compound of Formula III, embodiments comprising at least one of the steps:

a) Reacting a compound of Formula VIA or VIB with a nitrogen nucleophile, such as benzylamine (where P=benzyl) in an organic solvent, to afford a compound of Formula VA or VB.

b) Reacting a compound of Formula VIA or VIB with a compound of Formula VB or VA to afford a compound of Formula IV.

c) Optionally, deprotecting a mixture containing diastereomeric pairs of a compound of Formula IV (N-protected when P≠H) to afford a compound of Formula III.

A general preparation of nebivolol acid salts according to another embodiment of the present application proceeds as shown in FIG. 6, where P is a protecting group and HX is an acid.

According to an aspect of the application, there are provided processes for preparing the compound of Formula I, embodiments comprising at least one of the steps:

a) Reacting a compound of Formula VIA or VIB with a nitrogen nucleophile, such as benzylamine (where P=benzyl), optionally in the presence of a solvent, to afford a compound of Formula VA or VB.

b) Reacting a compound of Formula VIA or VIB with a compound of Formula VB or VA, optionally in the presence of a solvent, to afford a compound of Formula IV.

c) Subjecting the compound of Formula IV to purification under conditions appropriate for precipitation or crystallization of the compound of Formula IIIA having enhanced isomeric purity.

d) Deprotecting the compound of Formula IIIA of enhanced isomeric purity (N-protected when P≠H), to afford a compound of Formula IIA having enhanced isomeric purity.

e) Treating the compound of Formula IIA obtained in step d) with a suitable acid to afford the compound of Formula I.

According to an aspect of the application, there are provided processes for preparing the compound of Formula IIIA having enhanced purity, embodiments comprising at least one of the steps:

a) Reacting a compound of Formula VIA or VIB with a compound of Formula VB or VA, optionally in the presence of a solvent, to afford a compound of Formula IV.

b) Subjecting the compound of Formula IV to purification, resulting in precipitating or crystallizing a compound of Formula IIIA of enhanced isomeric purity.

An aspect of the present application provides processes for preparation of a compound of Formulas VIA or VIB, embodiments comprising at least one of the steps:

a) Reacting 6-fluorochroman-carboxylic acid with a reducing agent in a solvent, to afford 6-fluorochromamethanol (Formula VIII).

b) Treating a compound of Formula VIII with a radical initiator, in the presence of a halogen and a base, to afford 6-fluorochroman-carboxaldehyde (Formula VII).

c) Reacting a compound of Formula VII with trimethylsulfoxonium iodide in the presence of a base and solvent, to afford a compound of Formula VI.

d) Optionally, subjecting a compound of Formula VI to chromatographic purification, to afford the compounds of Formulas VIA and VIB.

In an aspect, the present application provides processes for the preparation of uniform and small particle sizes of nebivolol hydrochloride, embodiments comprising:

a) providing a solution of nebivolol hydrochloride in a solvent;

b) adding the mixture of step a) to an anti-solvent; and

c) recovering solid nebivolol hydrochloride.

An aspect of the present application provides pharmaceutical compositions comprising nebivolol.

### BRIEF DESCRIPTION OF THE DRAWINGS

FIG. 1 is an illustration of a powder X-ray diffraction (PXRD) pattern of a compound prepared according to Example 6.

FIG. 2 is an illustration of a differential scanning calorimetry (DSC) curve of a compound prepared according to Example 6.

FIG. 3 is an illustration of a PXRD pattern of crystalline Nebivolol hydrochloride prepared according to Example 8.

FIG. 4 is an illustration of a DSC curve of crystalline Nebivolol hydrochloride prepared according to Example 8.

FIG. 5 shows a reaction scheme for preparing nebivolol.

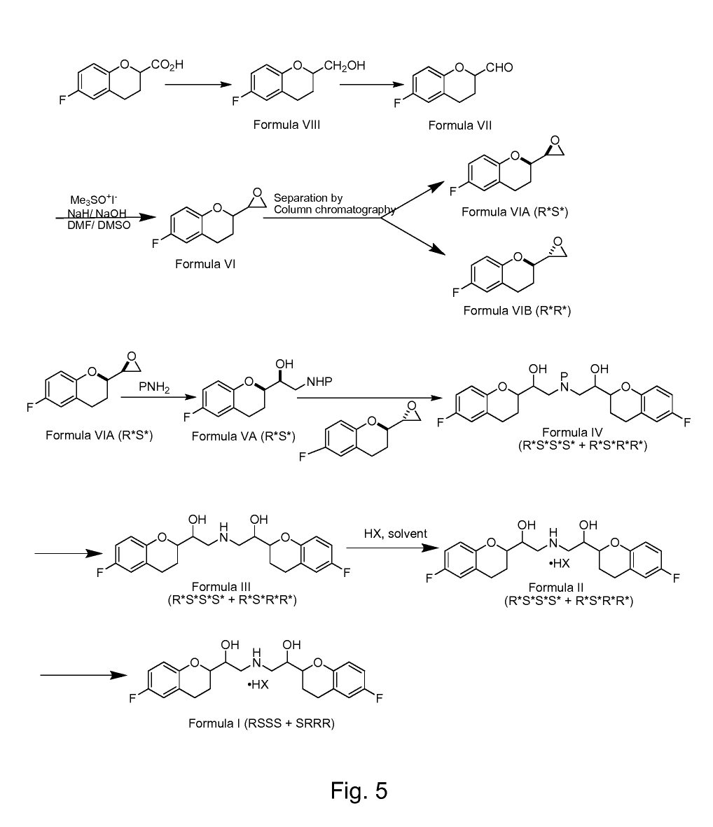

FIG. 6 shows a reaction scheme for preparing nebivolol.

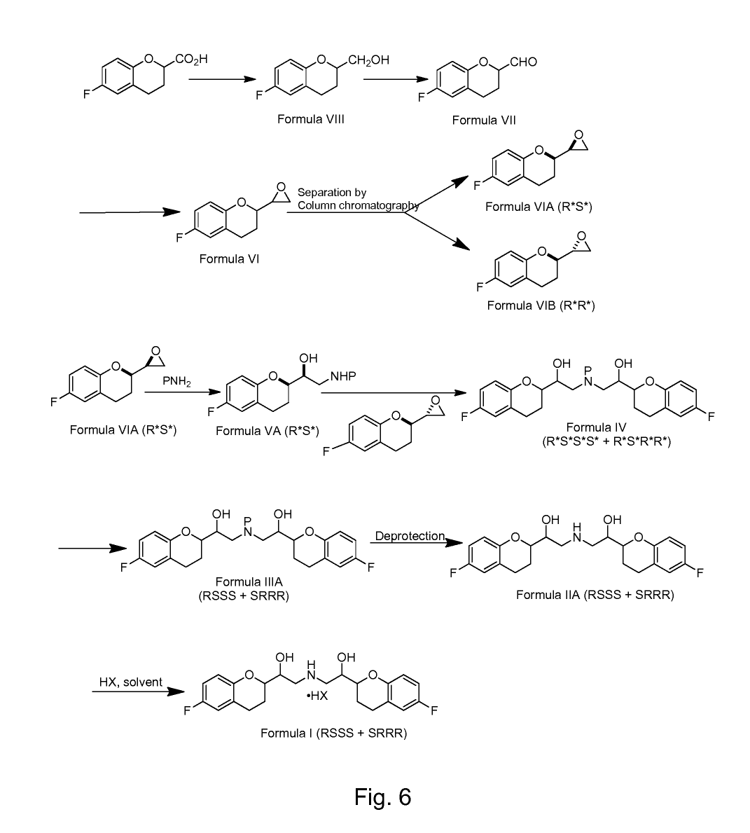

### DETAILED DESCRIPTION

In an aspect, there are provided processes for the preparation of nebivolol, including derivatives thereof such as salts, embodiments comprising at least one of the steps:

a) Reacting a compound of Formulas III or III′ with an acid in an organic solvent, to afford a compound of Formulas II or II′.

b) Purifying a compound of Formulas II or II′ in an organic solvent, or aqueous organic solvent, to afford a compound of Formula I, and optionally converting a compound of Formula Ito a hydrochloride salt, if HX in Formula I is not HCl.

Step a) involves preparation of an acid addition salt of a compound of Formulas III or III′, to afford a compound of Formulas II or II′.

Crystallization of a compound of Formula III or III′ (free base) can provide higher purity of the subsequent acid addition salt.

The acid addition salts can be prepared by reacting a mixture containing the compounds of Formula III or III′ with a desired acid in a suitable solvent. Acid addition salts are typically pharmaceutically acceptable, non-toxic addition salts with suitable acids, including, but not limited to: inorganic acids such as hydrohalogenic acids (for example, hydrofluoric, hydrochloric, hydrobromic, and hydroiodic acids) or other inorganic acids (for example, nitric, perchloric, sulphuric, and phosphoric acids); organic acids, such as organic carboxylic acids (for example, xinafoic, oxalic, propionic, butyric, glycolic, lactic, mandelic, citric, acetic, benzoic, 2- or 4-methoxybenzoic, 2- or 4-hydroxybenzoic, 2- or 4-chlorobenzoic, salicylic, succinic, malic, hydroxysuccinic, tartaric, fumaric, maleic, hydroxymaleic, oleic, and glutaric acids), organic sulphonic acids (for example, methanesulphonic, trifluoromethanesulphonic, ethanesulphonic, 2-hydroxyethanesulphonic, benzenesulphonic, toluene-p-sulphonic, naphthalene-2-sulphonic and camphorsulphonic acids), and amino acids (for example, ornithinic, glutamic, and aspartic acids).

Step a) is typically carried out in an organic solvent. Useful solvents include, but are not limited to: alcohols such as methanol, ethanol, isopropyl alcohol, and n-butanol; ketones such as 2-butanone; esters such as ethyl acetate, n-propyl acetate, and isopropyl acetate; acetonitrile; ethers such as tert-butyl methyl ether, diethyl ether, 1,4-dioxane, and tetrahydrofuran; aromatic hydrocarbons; C₁₋₅ halogenated hydrocarbons; aprotic polar solvents, such as N,N-dimethylformamide (DMF), dimethylsulfoxide (DMSO), and N,N-dimethylacetamide (DMA); water; and any mixtures of two or more thereof.

Suitable temperatures for the reaction may be less than about 100° C., or less than about 80° C., or less than about 60° C., or any other suitable temperatures.

Suitable times for this step may be from about 30 minutes to about 10 hours, or longer.

The acid addition salts of Formulas II or II′ have potential to serve as intermediates in the purification of nebivolol free base, or preparation of other acid addition salts by salt inter-conversion.

Step b) of the process involves purification of a compound of Formulas II or II′.

Compounds of Formulas II or II′ may be purified by precipitation or slurrying in suitable solvents, or by commonly known recrystallization techniques. The suitable recrystallization techniques include, but are not limited to, steps of concentrating, cooling, stirring, or shaking a solution containing the compound, combination of a solution containing a compound with an anti-solvent, seeding and partial removal of the solvent, or combinations thereof, evaporation, flash evaporation, and the like. An anti-solvent as used herein refers to a liquid in which a compound of Formula I is poorly soluble. Compounds of Formula II or II′ can be subjected to any of the purification techniques more than one time, until the desired purity for a compound of Formula I is attained.

Evaporation as used herein refers to distilling of solvent completely, or almost completely, at atmospheric pressure, or under reduced pressure. Flash evaporation as used herein refers to distilling of solvent using a technique such as, but not limited to, tray drying, spray drying, fluidized bed drying, and thin film drying, under reduced pressure or at atmospheric pressure.

The solvents that can be employed for crystallization or recrystallization include, but are not limited to: lower alcohols, such as methanol, ethanol, isopropyl alcohol, hexafluoroisopropyl alcohol, and 2,2,2-trifluoroethanol (TFE); ketones such as 2-butanone; esters such as ethyl acetate, n-propyl acetate, and isopropyl acetate; nitriles such as acetonitrile; ethers such as tert-butyl methyl ether, diethyl ether, 1,4-dioxane, and tetrahydrofuran; aromatic hydrocarbons; C₁₋₅ halogenated hydrocarbons; aprotic polar solvents such as N,N-dimethylformamide (DMF), dimethylsulfoxide (DMSO), and N,N-dimethylacetamide (DMA); water; and any mixtures of two or more thereof.

Step b) may be conduct for any desired time periods to achieve the desired product yield and purity. For example, times for step b) may vary from about 30 minutes to about 10 hours, or longer.

A purification process described above may be a recrystallization method for the removal of an undesired diastereomeric pair from a desired diastereomeric pair. The purification/recrystallization processes can be repeated until the separation of the desired racemic compound is obtained in a desired isomeric purity.

The preparation of nebivolol hydrochloride from a compound of Formula I (if the acid addition salt was not made with HCl) can be accomplished either by precipitating the free base from the acid addition salt of Formula I (other than with HCl) and its subsequent treatment with a suitable source of chloride ion, or by directly reacting the other acid addition salt with a source of chloride ion. Suitable sources of chloride ion for use in step (b) include, but are not limited to, hydrochloric acid.

According an aspect of the present application, there are provided processes for preparing a compound of Formula III, embodiments comprising at least one of the steps:

a) Reacting a compound of Formulas VIA or VIB with a nitrogen nucleophile, optionally in an organic solvent, to afford a compound of Formulas VA or VB.

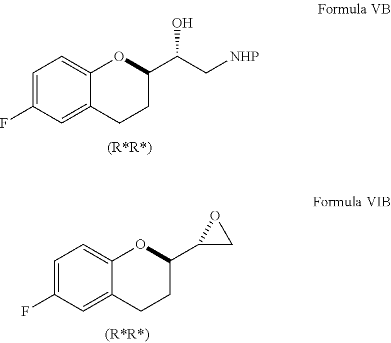

b) Reacting a compound of Formulas VIA or VIB with a compound of Formulas VB or VA, to afford a compound of Formula IV.

c) Treating a mixture containing diastereomeric pairs of a compound of Formula IV (N-protected) under deprotection conditions, to afford a compound of Formula III.

Step a) involves preparation of compound of Formulas VA or VB, by reacting a compound of Formula VIA or VIB with a nitrogen nucleophile, optionally in a solvent.

Suitable nucleophiles or protecting groups according to the application include nitrogen nucleophiles such as, but not limited to, azides, imides, amides, ammonia, hydrazine, carbamates, and amines. Some suitable protecting groups are disclosed in T. W. Greene et al., Protective Groups in Organic Synthesis, 3rd Ed., John Wiley & Sons, Inc., 1999, and other groups are described in the literature. In embodiments, the nucleophile comprises an amine. In specific embodiments, the nucleophile comprises benzylamine or ammonia.

Generally, the reaction is carried out in a solvent that is inert to the reaction conditions such as ethers, alcohols, esters, ketones, aromatic or aliphatic hydrocarbons, halogenated aliphatic or aromatic hydrocarbons, and any mixtures of two or more thereof. Specific examples of useful solvents are alcohols such as methanol, ethanol, isopropanol, etc. In embodiments, the reaction is carried out under neat conditions, without using any solvent.

Suitable temperatures for conducting the reaction range from about 0° C. to about the reflux temperature of the solvent used.

The reaction may be carried out for any desired time periods to achieve the desired product yield and purity. Typical reaction times vary from about 30 minutes to about 10 hours, or longer.

The compound of Formula VA or VB can optionally be isolated from the reaction mixture, such as by distillation of the reaction mixture (if a solvent is employed) followed by routine work-up, or by addition of a suitable organic solvent or aqueous organic solvent, or mixtures thereof, to the reaction mixture, if the reaction is carried out under neat conditions. Alternatively, the residue obtained by evaporation of reaction mixture can be progressed to a subsequent reaction step without any purification or work-up.

Optionally step a) may be carried out in the presence of boric acid or its derivatives.

Step b) involves reacting a compound of Formulas VIA or VIB with a compound of Formulas VB or VA, to afford a compound of Formula IV.

Step b) of the reaction may be carried out in solvents inert to the reaction conditions including, but not limited to: alcohols, such as, for example, methanol, ethanol, and isopropanol; ethers, such as, for example, 1,4-dioxane, tetrahydrofuran (THF), and methyl THF; esters, such as, for example, ethyl acetate, isopropyl acetate, and t-butyl acetate; halogenated hydrocarbons, such as, for example, dichloromethane, dichloroethane, chloroform, and the like; hydrocarbons, such as, for example, toluene, xylene, cyclohexane, and the like; acetic acid; and any mixtures of two or more thereof.

Suitable temperatures for conducting the reaction range from about 0° C. to about the reflux temperature of the solvent used.

Optionally, step b) may be carried out in the presence of boric acid or its derivatives.

Step c) involves preparing a compound of Formula III by deprotecting an N-protected compound of Formula IV (where P≠H).

The deprotection can be carried out under acidic, basic, or neutral conditions, depending on the nature of the protecting group employed. If a protecting group is benzyl, then its removal may be achieved using techniques that are generally known in the art. For example, it may be achieved by hydrogenation in the presence of a catalyst such as, for example, palladium or platinum on carbon, nickel on carbon, and palladium hydroxide on carbon, with hydrogen gas or a hydrogen source, e.g., ammonium formate, ammonium acetate, hydrazine, cyclohexadiene, or any other hydrogen source and a catalyst. The amount of catalyst employed may be 1-100%, or 5-50%, or 5-25%, of the weight of the compound of Formula IV.

The reaction may be carried out in solvents inert to the reaction conditions including, but not limited to: alcohols, such as, for example, methanol, ethanol, and isopropanol; ethers, such as, for example, 1,4-dioxane, THF, and methyl THF; esters, such as, for example, ethyl acetate, isopropyl acetate, and t-butyl acetate; halogenated hydrocarbons, such as, for example, dichloromethane, dichloroethane, chloroform, and the like; hydrocarbons, such as, for example, toluene, xylene, cyclohexane, and the like; acetic acid; and any mixtures of two or more thereof.

Hydrogen gas pressures for conducting the reaction may be about 1-100 Kg/cm², or 1-25 Kg/cm², or 1-10 Kg/cm². The reaction times will vary, depending on the activity of the catalyst and the amount that is used. Suitable times for the hydrogenation step may be from about 30 minutes to about 10 hours, or longer.

Suitable temperatures for the reaction may be less than about 200° C., or less than about 150° C., or less than about 100° C., or less than about 60° C., or any other suitable temperatures.

Optionally, the compound of Formula II′ can be prepared by reacting a compound of Formula VI with any suitable nitrogen nucleophile, such as benzylamine (P=benzyl) or ammonia (P═H), to afford a compound of Formula IV′ (P=benzyl) or Formula III′ (P═H), which optionally (when P≠H) on subsequent deprotection and salt formation yields a compound of Formula II′. In that case, a compound of Formula II′ and III′ will be a mixture of more than 4 isomers and may be purified by any of the recrystallization methods disclosed above, to afford nebivolol or any of its salts.

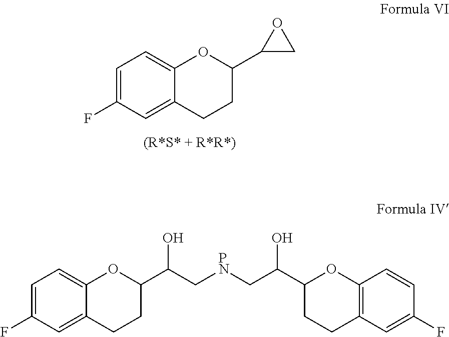

According to an aspect of the application, there are provided processes for preparation of a compound of Formula I, embodiments comprising at least one of the steps:

a) Reacting a compound of Formula VIA or VIB with a nitrogen nucleophile, optionally in the presence of a solvent, to afford a compound of Formula VA or VB.

b) Reacting a compound of Formula VIA or VIB with a compound of Formula VB or VA, optionally in the presence of a solvent, to afford a compound of Formula IV.

c) Subjecting the compound of Formula IV to purification by precipitation or crystallization to obtain the compound of Formula IIIA having enhanced isomeric purity.

d) Deprotecting the compound of Formula IIIA of enhanced isomeric purity (N-protected when P≠H) to afford a compound of Formula IIA having enhanced isomeric purity.

e) Treating the compound of Formula IIA obtained in step d) with a suitable acid to afford a compound of Formula I.

Step a) involves the preparation of a compound of Formula VA or VB, by reacting a compound of Formula VIA or VIB with a nitrogen nucleophile, optionally in a suitable solvent. This is similar to the reaction discussed above for forming a compound of Formula VA or VB.

Step b) of the process involves reacting a compound of Formula VIA or VIB with a compound of Formula VB or VA, to afford a compound of Formula IV. This is similar to the reaction discussed above for forming a compound of Formula IV.

Step c) of the process involves purification of the compound of Formula IV to afford a compound of Formula IIIA having enhanced isomeric purity.

A purification process may include precipitation or slurrying in suitable solvents, or commonly known crystallization/recrystallization techniques for the selective removal of an undesired diastereomeric pair from mixture of diastereomeric pairs. The purification/crystallization processes can be repeated until the separation of the desired racemic compound is obtained in a desired isomeric purity.

Suitable crystallization/recrystallization techniques include, but are not limited to, steps of concentrating, cooling, stirring, or shaking a solution containing the compound, combination of a solution containing a compound with an anti-solvent, seeding and partial removal of the solvent, or combinations thereof, evaporation, flash evaporation, and the like. An anti-solvent as used herein refers to a liquid in which a desired diastereomeric pair present in compound of Formula IV is poorly soluble. Compounds of Formula IV can be subjected to any of the purification technique more than one time until the desired isomeric purity of the compound of Formula IIIA is attained.

Evaporation as used herein refers to distilling of solvent completely, or almost completely, at atmospheric pressure, or under reduced pressure. Flash evaporation as used herein refers to distilling of solvent using a technique such as, but not limited to, tray drying, spray drying, fluidized bed drying, and thin film drying, under reduced pressure or at atmospheric pressure.

A compound of Formula IV may also be purified by precipitation or slurrying in suitable solvents, for example, by providing a mixture of diastereomeric pairs of compound of Formula IV in a suitable solvent, if required heating the resulting mixture to higher temperatures, subsequent cooling, and recovery of compound of Formula IIIA of enhanced isomeric purity. Alternatively, a reaction mixture comprising a solvent and the compound of Formula IV can be stirred at room temperature or lower temperatures for suitable times to result in effective precipitation of a desired diastereomer. The slurry can optionally be heated to temperatures about 40° C. to about reflux. To induce precipitation, the slurry is then cooled to about 0° C. to about room temperature, or to about 0° C. to about 5° C. Optionally, precipitation or crystallization at any of the above steps can be initiated by seeding of the reaction mixture with a small quantity of the desired product.

To ease the filtration of product, step-wise cooling with optional seeding can be performed. For example, a slurry comprising the compound of Formula IV can first be maintained at 40-50° C., followed by cooling to and stirring at 20-30° C., and subsequent cooling to and stirring at 0-10° C.

The solvents that can be employed for slurrying, crystallization, and recrystallization include, but are not limited to: lower alcohols, such as methanol, ethanol, isopropyl alcohol, hexafluoroisopropyl alcohol, and 2,2,2-trifluoroethanol (TFE); ketones such as 2-butanone; esters such as ethyl acetate, n-propyl acetate, and isopropyl acetate; nitriles such as acetonitrile; ethers such as tert-butyl methyl ether, diethyl ether, 1,4-dioxane, and tetrahydrofuran; aromatic or aliphatic hydrocarbons; C₁₋₅ halogenated hydrocarbons; aprotic polar solvents such as N,N-dimethylformamide (DMF), dimethylsulfoxide (DMSO), and N,N-dimethylacetamide (DMA); water; and any mixtures of two or more thereof.

The heating temperature is typically about 40 to 80° C., or the reflux temperature of the solvent. The cooling temperature is typically about 0-50° C., or about 10-15° C. The crystallization/precipitation time can be about 4-24 hours, or longer.

In embodiments, volume ratios of solvent to substrate are about 1:1 to 50:1.

In embodiments, steps b) and c) can be carried out in a single vessel.

In embodiments, steps a), b), and c) can be carried out in a single vessel.

Step d) involves preparing a compound of Formula IIA with enhanced purity, by deprotecting an N-protected compound of Formula IIIA (where P≠H) as obtained in step c).

The deprotection can be carried out under acidic, basic, or neutral conditions, depending on the nature of the protecting group employed. If a protecting group is benzyl, then its removal may be achieved using general techniques known in the art. For example, it may be achieved by hydrogenation in the presence of a catalyst such as, for example, palladium or platinum on carbon, nickel on carbon, and palladium hydroxide on carbon, with hydrogen gas or a hydrogen source, e.g., ammonium formate, ammonium acetate, hydrazine, cyclohexadiene, or any other hydrogen source and a catalyst. The amount of catalyst employed may be 1-100%, or 5-50%, or 5-25%, of the weight of the compound of Formula IIIA.

It has now surprisingly been found that it the amount of catalyst that is used, when deprotection is conducted with hydrogen gas, influences the formation of an undesired dehalogenated by-product, i.e., “de-mono-F” nebivolol. When smaller amounts of catalyst are used, the formation of “de-mono-F” nebivolol can be successfully controlled while obtaining substantially quantitative conversion to the debenzylated product. Thus, embodiments of the present application provide a simple and efficient method for deprotecting the intermediate, using hydrogen gas, that overcomes disadvantages associated with prior methods, by the use of less than 5% of a catalyst, such as 5% or 10% Pd on carbon, based on the weight of the compound of Formula IIIA. In specific embodiments, amounts of catalyst at least 1%, but less than 5%, 4%, 3%, or 2%, based on the weight of the compound of Formula IIIA, are contemplated.

The deprotection reaction may be carried out in solvents that are inert to the reaction conditions, including, but not limited to: alcohols, such as, for example, methanol, ethanol, 2-propanol, or 2-methoxyethanol; ethers, such as, for example, 1,4-dioxane, THF, and methyl THF; esters, such as, for example, ethyl acetate, isopropyl acetate, and t-butyl acetate; halogenated hydrocarbons, such as, for example, dichloromethane, dichloroethane, chloroform, and the like; hydrocarbons, such as, for example, toluene, xylene, cyclohexane, and the like; acetic acid; water and any mixtures thereof in various proportions. In specific embodiments, methanol is used as a reaction solvent.

Palladium-based catalysts useful for the debenzylation object of the application include 5% or 10% Pd on carbon (Pd/C), used either dry or wet. In specific embodiments, the Pd-based catalysts are combined with up to about 50% by weight water.

In embodiments, the amount of catalyst employed ranges from 1-5% of the weight of the compound of Formula IIIA. In embodiments, the amount of catalyst is about 1 to less than 2%, or less than 3%, of the weight of the compound of Formula IIIA.

In embodiments, hydrogen gas is used as a source of hydrogen. Hydrogen gas pressures for conducting the reaction may vary from about 1-100 Kg/cm², or about 1-25 Kg/cm², or about 1-10 Kg/cm², or about 1.2 Kg/cm². The reaction times will vary depending on the activity of the catalyst and the amounts of catalyst and reactants used.

Suitable temperatures for the reaction may be less than about 200° C., or less than about 150° C., or less than about 100° C., or less than about 60° C., or any other suitable temperatures.

The times for completion of reaction, with smaller amounts of catalyst in the case of catalytic hydrogenation, has been experienced to be less than that for catalytic transfer hydrogenation (CTH).

Suitable times for the hydrogenation step may be from about 30 minutes to about 10 hours, or longer.

Step e) involves reacting the compound of Formula IIA obtained in step d) with a suitable acid, to afford the compound of Formula I.

The compound of Formula IIA can optionally be purified before subjecting it to a reaction with an acid. A purification process that could be employed may be a recrystallization method for further removal of an undesired diastereomeric pair from a desired diastereomeric pair. The purification, such as recrystallization, processes can be repeated until the desired racemic compound is obtained in a desired isomeric purity.

A compound of Formula IIA can also be purified by preparation of its acid addition salt of Formula I followed by its crystallization and, if desired, subsequent liberation of free base followed by acid salt preparation. If desired, nebivolol hydrochloride (Formula I, HX=HCl) can directly be prepared from another acid addition salt of Formula I (when X≠Cl) by salt inter-conversion.

Suitable acids that can be employed in step e) include, but are not limited to, inorganic acids such as hydrochloric acid, sulphuric acid, and phosphoric acid, and organic acids such as methanesulfonic acid, benzenesulfonic acid, propionic acid, etc.

The acids employed can be in the form of aqueous solutions or alcohol solutions, or can be used in gaseous form wherever applicable, to afford the acid addition salt of nebivolol. For example, hydrogen chloride gas can be passed through a mixture comprising nebivolol free base, to afford the nebivolol hydrochloride salt with enhanced purity.

Nebivolol hydrochloride can be prepared from the compound obtained in step d) either by isolation of the compound therein or by treating the filtrate obtained by filtration of a reaction mixture of step d) with a source of chloride ion.

Suitable sources of chloride ion for use in step e) include, but are not limited to, hydrochloric acid.

The preparation of nebivolol hydrochloride from a compound of Formula I (if the acid addition salt was not made with HCl) can be accomplished either by forming the free base from the acid addition salt of Formula I (other than with HCl) and its subsequent treatment with a suitable source of chloride ion, or by directly reacting the other acid addition salt with a source of chloride ion. Suitable sources of chloride ion for use in step d) include, but are not limited to, hydrochloric acid.

According to an aspect of the application, there are provided processes for preparing the compound of Formula IIIA having enhanced purity, embodiments comprising at least one of the steps:

a) Reacting a compound of Formula VIA or VIB with a compound of Formula VB or VA, optionally in the presence of a solvent, to afford a compound of Formula IV.

b) Subjecting the compound of Formula IV to purification, under conditions resulting in precipitation or crystallization of a compound of Formula IIIA of enhanced isomeric purity.

Step a) may be carried out in solvents that are inert to the reaction conditions including, but not limited to: alcohols, such as, for example, methanol, ethanol, and isopropanol; ethers, such as, for example, 1,4-dioxane, tetrahydrofuran (THF), and methyl THF; esters, such as, for example, ethyl acetate, isopropyl acetate, and t-butyl acetate; halogenated hydrocarbons, such as, for example, dichloromethane, dichloroethane, chloroform, and the like; hydrocarbons, such as, for example, toluene, xylene, cyclohexane, and the like; acetic acid; and any mixtures of two or more thereof. Also, the reaction can be carried under neat conditions, without a solvent.

Suitable temperatures for conducting the reaction range from about 0° C. to about the reflux temperature of the solvent used.

Step b) involves purification of a compound of Formula IV to afford a compound of Formula IIIA having enhanced isomeric purity.

A purification process may include precipitation or slurrying in suitable solvents, or commonly known crystallization/recrystallization techniques for the selective removal of an undesired diastereomeric pair from mixture of diastereomeric pairs. The purification or crystallization/recrystallization processes can be repeated until the desired racemic compound is obtained in a desired isomeric purity.

Suitable crystallization/recrystallization techniques include, but are not limited to, steps involving concentrating, cooling, stirring, or shaking a solution containing the compound, combination of a solution containing a compound with an anti-solvent, seeding and partial removal of the solvent, or combinations thereof, evaporation, flash evaporation, and the like. An anti-solvent as used herein refers to a liquid in which desired diastereomeric pair present in compound of Formula IV is poorly soluble. Compounds of Formula IV can be subjected to any of the purification techniques more than one time until the desired isomeric purity of the compound of Formula IIIA is attained.

Evaporation as used herein refers to distilling of solvent completely or almost completely, at atmospheric pressure, or under reduced pressure. Flash evaporation as used herein refers to distilling of solvent using techniques such as, but not limited to, tray drying, spray drying, fluidized bed drying, and thin film drying, under reduced pressure or at atmospheric pressure.

Compounds of Formula IV may also be purified by precipitation or slurrying in suitable solvents. For example, a mixture of diastereomeric pairs of the compound of Formula IV may be dissolved in a suitable solvent, if required heating the mixture to higher temperatures, and subsequently cooling and recovering the compound of Formula IIIA with enhanced isomeric purity. Alternatively, a reaction mixture comprising a solvent and the compound of Formula IV, upon stirring at room temperature or lower temperatures, may result in precipitation of the desired compound of Formula IIIA. A slurry can optionally be heated to temperatures about 40° C. to about reflux. To induce precipitation, the slurry is then cooled to about 0° C. to about room temperature, or about 0° C. to about 5° C.

Optionally, precipitation or crystallization by any of the above steps can be initiated by seeding of the reaction mixture with a small quantity of the desired product.

To facilitate filtration of the product, step-wise cooling with optional seeding can be performed. For example, a slurry comprising the compound of Formula IV can first be maintained at 40-50° C., followed by cooling and stirring at 20-30° C., and subsequent cooling with stirring at 0-10° C.

The solvents that can be employed for slurrying, crystallization, and recrystallization include, but are not limited to: lower alcohols, such as methanol, ethanol, isopropyl alcohol, hexafluoroisopropyl alcohol, and 2,2,2-trifluoroethanol (TFE); ketones such as 2-butanone; esters such as ethyl acetate, n-propyl acetate, and isopropyl acetate; nitriles such as acetonitrile; ethers such as tert-butyl methyl ether, diethyl ether, 1,4-dioxane, and tetrahydrofuran; aromatic hydrocarbons; C₁₋₅ halogenated hydrocarbons; aprotic polar solvents such as N,N-dimethylformamide (DMF), dimethylsulfoxide (DMSO), and N,N-dimethylacetamide (DMA); water; and any mixtures of two or more thereof.

The temperatures from heating can be about 40-80° C., or the reflux temperature of the solvent. The temperatures from cooling can be about 0-50° C., or about 10-15° C. The crystallization/precipitation times can be about 4-24 hours, or longer.

In embodiments, volume ratios of solvent to substrate are about 1:1 to 50:1.

In embodiments, steps a) and b) can be carried out in a single vessel, without isolation of an intermediate product.

An aspect of the application provides processes for preparing a compound of Formulas VIA or VIB, embodiments comprising at least one of the steps:

a) Reacting 6-fluorochroman-2-carboxylic acid with a reducing agent in a solvent, to afford 6-fluorochroman-2-yl-methanol (Formula VIII).

b) Treating a compound of Formula VIII with a radical initiator in the presence of an oxidizing agent and a base, to afford 6-fluorochroman-carboxaldehyde (Formula VII).

c) Reacting a compound of Formula VII with trimethylsulfoxonium iodide in the presence of a base and a solvent, to afford a compound of Formula VI.

d) Optionally, subjecting a compound of Formula VI to chromatographic purification to afford compounds of Formulas VIA and VIB.

Step a) of the process involves reduction of 6-fluorochroman-2-carboxylic acid to afford 6-fluorochroman-2-yl-methanol of Formula VIII.

Suitable reducing agents employed in step b) include, but are not limited to, lithium aluminum hydride, NaBH₄, and Vitride™ (a solution of ≧65% by wt. of sodium bis(2-methoxyethoxy)aluminum hydride in toluene).

Step a) can be carried out in the presence of an acid catalyst such as sulfuric acid, boric acid, boronic acid, etc.

Amounts of acid catalyst used are in the range of about 0.1-1 molar equivalent of per mole of 6-fluorochroman-2-carboxylic.

The reaction may be suitably carried out in solvents that are inert to the reaction conditions, including: alcohols such as methanol, ethanol, and isopropanol; ethers such as 1,4-dioxane and THF; halogenated hydrocarbons such as dichloromethane, dichloroethane, chloroform, and the like; aromatic hydrocarbons such as toluene, xylene, cyclohexane, and the like; acetic acid; and any mixtures of two or more thereof.

Suitable temperatures for conducting the reaction range from about 10° C. to about the reflux temperature of the solvent used.

The reaction may be carried out for any desired time periods to achieve the desired product yield and purity. The reaction times vary from about 30 minutes to about 10 hours, or longer.

Step b) involves oxidation of a compound of Formula VIII, to afford 6-fluorochroman-carboxaldehyde (Formula VII).

Useful oxidizing agents for step b) include, but are not limited to, halogens, such as iodine. The reaction is conducted in the presence of a radical initiator such as 2,2,6,6-tetramethylpiperidine-1-oxyl (TEMPO) or a derivative thereof, and a base.

Amounts of halogen used are in the range of about 0.1-4, or about 1, molar equivalent of iodine per mole of a compound of Formula VIII.

Amounts of radical initiator used are in the range of about 0.01-1.5 molar equivalents, per mole of a compound of a Formula VIII.

Amounts of base used are in the range of about 0.5-5 molar equivalents, per mole of a compound of Formula VIII.

Suitable bases that can be employed in step b) include, but are not limited to: inorganic bases such as metal hydroxides, alkoxides, carbonates, and bicarbonates; and organic bases such as pyridine, lutidine, triethylamine, 4-dimethylaminopyridine (DMAP), dicyclohexylamine, diisopropylethylamine, and the like. A specific example of a useful base is sodium bicarbonate, e.g., in an aqueous solution.

The oxidation is carried in a biphasic reaction medium comprising an organic solvent and aqueous base. Organic solvents that can be employed in step b) include, but are not limited to, halogenated hydrocarbons such as dichloromethane, dichloroethane, chloroform, and the like; hydrocarbons such as toluene, xylene, cyclohexane, and the like; aliphatic esters such as methyl acetate and ethyl acetate; ethers such as diethyl ether and diisopropyl ether; and any mixtures of two or more thereof.

Reactions are carried out at temperatures ranging from range from about 0° C. to about the reflux temperature of the solvent used. For example, the reaction can be performed at temperatures about 15-25° C.

The reaction times vary from about 30 minutes to about 24 hours, or longer.

Step c) involves oxirane formation, starting from a compound of Formula VII, by treatment with trimethylsulfoxonium iodide in the presence of a base and organic solvent.

The reaction may be suitably carried out in a solvent inert to the reaction conditions, including polar solvents such as dimethylsulfoxide, N,N-dimethylformamide, water, and any mixtures thereof.

The bases employed in step c) include, but are not limited to, inorganic bases such as sodium hydride, sodium hydroxide, potassium hydroxide, sodium carbonate, potassium carbonate, sodium bicarbonate, potassium bicarbonate, and the like.

Step d) involves separating diastereomeric pairs of 6-fluoro-2-(oxiran-2-yl)chroman (Formula VI), to recover (R*S*)-6-fluoro-2-(oxiran-2-yl)chroman (Formula VIA) as the A-isomer and (R*R*)-6-fluoro-2-(oxiran-2-yl)chroman (Formula VIB) as the B-isomer, using column chromatography.

The mixture of oxiranes can be separated by column chromatography to elute first a pure fraction of isomer A (Formula VIA), i.e., (A)-6-fluoro-3,4-dihydro-2-oxiranyl-2H-1-benzopyran of European Patent 0145067. Upon further elution, the second fraction (isomer B, i.e., Formula VIB), i.e., (B)-6-fluoro-3,4-dihydro-2-oxiranyl-2H-1-benzopyran of European Patent 0145067 is obtained. The chromatographic separation of isomers A and B is carried out using silica gel as the stationary phase and the eluant is a mixture of hexanes and ethyl acetate. If the loaded material is a large quantity, the purification by column chromatography can be done simultaneously on multiple columns. The column chromatography yields isomer A in the beginning, followed by isomer B. Based on the purities determined for fractions from the column, some of them can be mixed together and used for further reaction.

The solids at any stage of the processes of the present application may be recovered from a suspension or solution, using any of techniques such as decantation, filtration by gravity or by suction, centrifugation, slow evaporation, and the like, or any other suitable techniques. The solids that are isolated may carry a small proportion of occluded mother liquor containing a higher percentage of impurities. If desired, the solids may be washed with a solvent to wash out the mother liquor and/or impurities, and the resulting wet solids may optionally be dried. Evaporation, as used herein, refers to distilling of solvent completely or almost completely, at atmospheric pressure or under reduced pressure. Flash evaporation as used herein refers to distilling of solvent using techniques including, but not limited to, tray drying, spray drying, fluidized bed drying, and thin film drying, under reduced pressure or at atmospheric pressure.

A wet cake that is obtained may be optionally dried. Drying may be carried out using a tray dryer, vacuum oven, air oven, fluidized bed dryer, spin flash dryer, flash dryer, and the like, at atmospheric pressure or under reduced pressure. The drying may be carried out at temperatures less than about 200° C., or about 20° C. to about 80° C., or about 30° C. to about 60° C., or any other suitable temperatures, at atmospheric pressure or under reduced pressure. The drying may be carried out for any desired times until the desired quality of product is achieved, such as about 30 minutes to about 5 hours, or about 1 to about 4 hours. Shorter or longer times also are useful.

In an aspect, the present application provides processes for the preparation of small and uniform particle sizes and distributions of nebivolol hydrochloride, embodiments comprising:

a) providing a solution of nebivolol hydrochloride in a solvent;

b) combining the solution of step a) with an anti-solvent; and

c) recovering solid nebivolol hydrochloride.

Step a) involves providing a mixture of nebivolol hydrochloride in a solvent.

The solution comprising nebivolol hydrochloride in step a) may be prepared by dissolving nebivolol in a sufficient quantity of a solvent, forming a suspension of nebivolol in a solvent and heating to dissolve the solid, or using a solution resulting from a final step in the synthesis of nebivolol hydrochloride.

To obtain a clear solution of nebivolol hydrochloride it may be required to heat to elevated temperatures, provided that the stability of the nebivolol hydrochloride is not compromised and a substantially clear solution is obtained. For example, the dissolution temperatures may range from about 20° C. to about the reflux temperature of the solvent.

Suitable solvents for step a) include, but are not limited to: alcohols, such as, for example, methanol, ethanol, 2-propanol, n-butanol, and the like; ketones, such as, for example, acetone, ethyl methyl ketone, methyl isobutyl ketone, and the like; nitriles, such as, for example, acetonitrile, propionitrile, and the like; polar aprotic solvents such as N,N-dimethylformamide and dimethylsulfoxide; mixtures of any two or more thereof; and their combinations with water in various proportions.

A solution can optionally be filtered by passing through paper, glass fiber, or a membrane material, or a bed of a clarifying agent such as Celite™. Depending upon the equipment used and the concentration and temperature of the solution, the filtration apparatus may need to be heated to avoid premature crystallization.

Step b) involves combining the solution of step a) with an anti-solvent.

Suitable anti-solvents that are useful in this step include, but are not limited to, ethers such as diethyl ether, dimethyl ether, diisopropyl ether, methyl t-butyl ether, tetrahydrofuran, 1,4-dioxane, and the like; esters such as, ethyl acetate, n-propyl acetate, n-butyl acetate, and t-butyl acetate; hydrocarbons, such as heptane, hexane, toluene, xylene, and the like; halogenated hydrocarbons, such as, dichloromethane, chloroform, and the like; and any combinations of two or more thereof.

Suitable temperatures for combining an anti-solvent with a mixture of step a) range from about −20° C. to about 40° C. In embodiments, the anti-solvent is used in an amount about 0.25 times to about 5 times the volume of the solution containing nebivolol hydrochloride.

In embodiments, a solution of nebivolol hydrochloride is added to a stirred quantity of anti-solvent.

The mixture may be stirred for solid formation at temperatures such as, for example, about −20° C. to about 40° C., for times as required for solid formation. The exact temperatures and times required for complete solid formation can be readily determined by a person skilled in the art.

Step c) involves recovering nebivolol hydrochloride from step b).

The methods by which the solid material is recovered from the final mixture, with or without cooling below the operating temperature, can be any of techniques such as decantation, filtration by gravity or suction, centrifugation, and the like. The isolated solid may carry a small proportion of occluded mother liquor containing a higher percentage of impurities. If desired, the isolated solid may be washed with a solvent to wash out the mother liquor.

The wet cake obtained may optionally be further dried. Drying can be suitably carried out in a tray dryer, vacuum oven, air oven, fluidized bed dryer, spin flash dryer, flash dryer, and the like. The drying can be carried out at temperatures from about 30° C. to about 90° C., with or without applying vacuum. The drying can be carried out for any desired times until the desired product purity is achieved.

Nebivolol and its salts, such as the hydrochloride, and drug-related impurities may be analyzed using high performance liquid chromatography (HPLC), for example by a method using a CHIRALPAKTM AD-3, 250×4.6 mm×3 μm column, with the following parameters:

| | |
|---|---|
| Flow rate | 0.8 mL/minute. |
| Detector | 280 nm. |
| Column oven temp. | 40° C. |
| Injection volume | 20 μL. |
| Run time | 50 minutes. |
| Elution | Isocratic. |
| Mobile phase | A mixture of n-hexane, ethanol, isopropyl alcohol, |
| | and diethanolamine, respectively in a |
| | 420:450:130:1 volume ratio. |
| Diluent | Methanol. |
| Sample preparation | Dissolve 10 mg of sample in diluent and make up |
| | the volume to 10 mL with diluent. |

According an aspect of the present application, there are provided pharmaceutical compositions and processes for manufacturing pharmaceutical compositions, using nebivolol or a salt thereof of the present application.

Pharmaceutical compositions that include nebivolol or a salt thereof with one or more pharmaceutically acceptable excipients may be formulated as: solid oral dosage forms such as, but not limited to, powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the form of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir systems or combinations of matrix and reservoir systems. The compositions may be prepared by direct blending, dry granulation, and wet granulation or by extrusion and spheronization. Compositions may be presented as uncoated, film coated, sugar coated, powder coated, enteric coated or modified release coated. Compositions of the present application may further comprise one or more pharmaceutically acceptable excipients.

Pharmaceutically acceptable excipients that are useful for preparing formulations include, but are not limited to: diluents such as starch, pregelatinized starch, lactose, powdered celluloses, microcrystalline celluloses, dicalcium phosphate, tricalcium phosphate, mannitol, sorbitol, sugar, and the like; binders such as acacia, guar gum, tragacanth, gelatin, polyvinylpyrrolidones, hydroxypropyl celluloses, hydroxypropyl methylcelluloses, pregelatinized starch, and the like; disintegrants such as starch, sodium starch glycolate, pregelatinized starch, crospovidones, croscarmellose sodium, colloidal silicon dioxide, and the like; lubricants such as stearic acid, magnesium stearate, zinc stearate, and the like; glidants such as colloidal silicon dioxide and the like; solubility or wetting enhancers such as anionic or cationic or neutral surfactants; complex forming agents such as various grades of cyclodextrins and resins; release rate controlling agents such as hydroxypropyl celluloses, hydroxymethyl celluloses, hydroxypropyl methylcelluloses, ethylcelluloses, methylcelluloses, various grades of methyl methacrylates, waxes, and the like. Other pharmaceutically acceptable excipients that are of use include, but are not limited to, film formers, plasticizers, colorants, flavoring agents, sweeteners, viscosity enhancers, preservatives, antioxidants and the like.

The processes of present application are simple, cost-effective, ecologically friendly, reproducible, scalable, and robust, to produce nebivolol or its salts with high chemical and isomeric purity.

The compounds obtained by the present application, unless stated otherwise, can be characterized by their PXRD patterns and DSC curves, as well as by other common analytical techniques. PXRD data reported herein were obtained using copper Ka radiation, and were obtained using a Bruker AXS D8 Advance Powder X-ray Diffractometer. DSC analyses were carried out in a DSC Q1000 instrument from TA Instruments with a ramp of 10° C./minute, up to 250° C.

Crystalline forms can be characterized by scattering techniques, e.g., powder X-ray diffraction patterns, using spectroscopic methods, e.g., infrared absorption spectrophotometry, ¹³C nuclear magnetic resonance spectroscopy, and using thermal techniques, e.g., differential scanning calorimetry and differential thermal analysis. Generally, crystalline forms are best characterized by their X-ray powder diffraction patterns, determined in accordance with procedures that are known in the art. For a discussion of these techniques see J. Haleblain, Journal of Pharmaceutical Science, Vol. 64, pages 1269-1288 (1975), and J. Haleblain and W. McCrone, Journal of Pharmaceutical Science, Vol. 58, pages 911-929 (1969).

Generally, a diffraction angle (2θ) in powder X-ray diffractometry may have an error in the range of about ±0.2°. Therefore, the diffraction angle values should be understood as including values in the range of about ±0.2°. Accordingly, the present application includes not only crystals whose peak diffraction angles in powder X-ray diffractometry completely coincide with each other, but also crystals whose individual peak diffraction angles coincide with each other with the specified tolerance. Therefore, in the present specification, the phrase “having a diffraction peak at a diffraction angle (2θ±0.2°) of 7.9°” would mean “having a diffraction peak at a diffraction angle (2θ) of 7.7° to 8.1°”. Although the intensities of peaks in the x-ray powder diffraction patterns of different batches of a compound may vary slightly, the peaks and the peak locations are characteristic for a specific polymorphic form. Alternatively, the term “about” means within an acceptable standard error of the mean, when considered by one of ordinary skill in the art. The relative intensities of the PXRD peaks can vary depending on the sample preparation technique, crystal size distribution, various filters used, the sample mounting procedure, and the particular instrument employed. Moreover, instrument variation and other factors can affect the 2θ values. Therefore, the term “substantially” in the context of PXRD is meant to encompass that peak assignments can vary by plus or minus about 0.2 degrees. Moreover, new peaks may be observed or existing peaks may disappear, depending on the type of the machine or the settings (for example, whether a nickel filter is used or not).

The D₁₀, D₅₀, and D₉₀ values are useful ways for indicating particle size distributions. D₉₀ refers to at least 90 volume percent of the particles having a size smaller than the given value. Likewise, D₁₀ refers to 10 volume percent of the particles having a size smaller than the given value. D₅₀ refers to 50 volume percent of the particles having a size smaller than the given value. Methods for determining D₁₀, D₅₀, and D₉₀ include laser diffraction, such as using equipment from Malvern Instruments Ltd. of Malvern, Worcestershire, United Kingdom.

### DEFINITIONS

The following definitions are used in connection with the present application unless the context indicates otherwise. An “alcohol solvent” is an organic solvent containing a carbon bound to a hydroxyl group. “Alcohol solvents” include, but are not limited to, methanol, ethanol, 2-nitroethanol, 2-fluoroethanol, 2,2,2-trifluoroethanol, 1,1,1,3,3,3-hexafluoropropan-2-ol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol, and the like.

“Aliphatic hydrocarbon” refers to a liquid hydrocarbon, which may be linear or branched. Examples of aliphatic hydrocarbons include, but are not limited to, n-pentane, isopentane, neopentane, n-hexane, isohexane, 3-methylpentane, 2,3-dimethylbutane, neohexane, n-heptane, isoheptane, 3-methylhexane, neoheptane, 2,3-dimethylpentane, 2,4-dimethylpentane, 3,3-dimethylpentane, 3-ethylpentane, 2,2,3-trimethylbutane, n-octane, isooctane, 3-methylheptane, neooctane, C₅-C₈ aliphatic hydrocarbons, and mixtures thereof.

“Amine-protecting group” refers to a radical when attached to a nitrogen atom in a target molecule is capable of surviving subsequent chemical reactions applied to the target molecule, i.e., hydrogenation, reaction with acylating agents, alkylation etc. The amine-protecting group can later be removed. Amine protecting groups include, but are not limited to, fluorenylmethoxycarbonyl (FMOC), tert-butoxycarbonyl (t-BOC), benzyloxycarbonyl (Z), those of the acyl type (e.g., formyl, benzoyl, trifluoroacetyl, p-tosyl, aryl- or alkylphosphoryl, phenyl- or benzylsulfonyl, o-nitrophenylsulfenyl, o-nitrophenoxyacetyl), or of the urethane type (e.g. tosyloxyalkyloxy-, cyclopentyloxy-, cyclohexyloxy-, 1,1-dimethylpropyloxy, 2-(p-biphenyl)-2-propyloxy- or benzylthiocarbonyl). Amine-protecting groups are made using a reactive agent capable of transferring an amine-protecting group to a nitrogen atom in the target molecule. Examples of an amine-protecting agent include, but are not limited to, C₁-C₆ aliphatic acid chlorides or anhydrides, C₆-C₁₄ arylcarboxylic acid chlorides or anhydrides, t-butyl chloroformate, di-tert-butyl dicarbonate, butoxycarbonyloxyimino-2-phenylacetonitrile, t-butoxycarbonyl azide, t-butyl fluoroformate, fluorenylmethoxycarbonyl chloride, fluorenylmethoxycarbonyl azide, fluorenylmethoxycarbonyl benzotriazol-1-yl, (9-fluorenylmethoxycarbonyl) succinimidyl carbonate, fluorenylmethoxycarbonyl pentafluorophexoxide, trichloroacetyl chloride, methyl-, ethyl-, trichloromethyl-chloroformate, or other amine protecting agents known in the art. Examples of such known amine-protecting agents are found in pages 385-397 of T. W. Green, P. G. M. Wuts, Protective Groups in Organic Synthesis, Second Edition, Wiley-Interscience, New York, 1991.

“Aromatic hydrocarbon” refers to a liquid, unsaturated, cyclic, hydrocarbon containing one or more rings having at least one 6-carbon ring containing three double bonds. Examples of an aromatic hydrocarbons include, but are not limited to, benzene, toluene, ethylbenzene, m-xylene, o-xylene, p-xylene, indane, naphthalene, tetralin, trimethylbenzene, chlorobenzene, fluorobenzene, trifluorotoluene, anisole, C₆-C₁₀ aromatic hydrocarbons, and mixtures thereof.

Celite™ is flux-calcined diatomaceous earth. Celite™ is a registered trademark of World Minerals Inc.

An “ester” is an organic compound containing a carboxyl group —(C═O)—O— bonded to two other carbon atoms.

CHIRALPAK™ IA is amylose tris(3,5-dimethylphenylcarbamate) immobilized on silica gel. CHIRALPAK™ is a registered trademark of DAICEL CHEMICAL INDUSTRIES LTD.

“Hyflow” is a flux-calcined diatomaceous earth treated with sodium carbonate. Hyflo Super Cel™ is a registered trademark of the Manville Corp.

An “ether” is an organic compound containing an oxygen atom —O— onded to two other carbon atoms. “Ethers” include, but are not limited to, diethyl ether, diisopropyl ether, methyl t-butyl ether, glyme, diglyme, tetrahydrofuran, methyl tetrahydrofuran, 1,4-dioxane, and the like.

A “halogenated hydrocarbon” is an organic compound containing a carbon bound to a halogen. “Halogenated hydrocarbons” include, but are not limited to, dichloromethane, 1,2-dichloroethane, trichloroethylene, perchloroethylene, 1,1,1-trichloroethane, chloroform, carbon tetrachloride, and the like.

“Hydrocarbon” refers to a liquid hydrocarbon, which may be linear, branched, or cyclic. Examples of hydrocarbons include, but are not limited to, n-pentane, isopentane, neopentane, n-hexane, isohexane, 3-methylpentane, 2,3-dimethylbutane, neohexane, n-heptane, isoheptane, 3-methylhexane, neoheptane, 2,3-dimethylpentane, 2,4-dimethylpentane, 3,3-dimethylpentane, 3-ethylpentane, 2,2,3-trimethylbutane, n-octane, isooctane, 3-methylheptane, neooctane, C₅-C₈ aliphatic hydrocarbons, cyclohexane, methylcyclohexane, benzene, toluene, ethylbenzene, m-xylene, o-xylene, p-xylene, indane, naphthalene, tetralin, trimethylbenzene, C₆-C₁₀ aromatic hydrocarbons, and the like.

A “ketone” is an organic compound containing a carbonyl group —(C═O)— bonded to two other carbon atoms. “Ketones” include, but are not limited to, acetone, ethyl methyl ketone, diethyl ketone, methyl isobutyl ketone, and the like.

A “nitrile” is an organic compound containing a cyano —(C≡N) bonded to another carbon atom. “Nitriles” include, but are not limited to, acetonitrile, propionitrile, C₂₋₆ nitriles, and the like.

A “protecting group” is capable of surviving subsequent chemical reactions when applied to a target molecule, i.e., reactions such as hydrogenation, reaction with acylating agents, alkylation, etc. The protecting group can later be removed.

All percentages and ratios used herein are by weight of the total composition and all measurements made are at 25° C. and atmospheric pressure unless otherwise designated. All temperatures are in degrees Celsius unless specified otherwise. The present application can comprise (open ended) the components of the present application as well as other ingredients or elements described herein.

As used herein, “comprising” means the elements recited, or their equivalents in structure or function, plus any other element or elements which are not recited. The terms “having” or “including” are also to be construed as open ended unless the context suggests otherwise.

All ranges recited herein include the endpoints, including those that recite a range “between” two values.

Terms such as “about,” “generally,” “substantially,” or the like are to be construed as modifying a term or value such that it is not an absolute. Such terms will be defined by the circumstances and the terms that they modify as those terms are understood by those of skill in the art. This includes, at the very least, the degree of expected experimental error, technique error and instrument error for a given technique used to measure a value.

When a molecule or other material is identified herein as “pure”, it generally means, unless specified otherwise, that the material has 99% purity or greater, as determined by methods conventional in the art, such as high performance liquid chromatography (HPLC) or spectroscopic methods. In general, this refers to purity with regard to unwanted residual solvents, reaction by-products, impurities, or unreacted starting materials. In the case of stereoisomers, “pure” means 99% of one enantiomer or diastereomer, as appropriate. When a compound is identified herein as of “enhanced isomeric purity” or “substantially pure”, it generally means, unless specified otherwise, that the material has at least 85% purity or greater, with regard to unwanted isomers (one enantiomer or diastereomer, as appropriate), as determined by methods conventional in the art such as high performance liquid chromatography (HPLC) or spectroscopic methods.

The term “diastereomeric excess” (abbreviated “de”) shall mean the percentage of major diastereomer less the percentage of minor diastereomer, in a sample.

The processes of the present application provide a compound of structural Formula I with high diastereomeric purity, typically in excess of 99% de. In embodiments, a compound of Formula I is obtained with a diastereomeric purity of 99.5% de. In embodiments, a compound of Formula I is obtained with a diastereomeric purity of 99.9% de.

The chemical transformations described throughout the specification may be carried out using substantially stoichiometric amounts of reactants, though certain reactions may benefit from using an excess of one or more of the reactants. Additionally, many of the reactions disclosed throughout the specification may be carried out at room temperature, but particular reactions may require the use of higher or lower temperatures, depending on reaction kinetics, yields, or the like. Furthermore, many of the reactions may employ one or more compatible solvents, which may influence the reaction rate or yield. Depending on the nature of the reactants, one or more solvents that may be polar protic solvents, polar aprotic solvents, non-polar solvents, or some combinations can be employed.

Certain specific aspects and embodiments of the present disclosure will be explained in more detail with reference to the following examples, which are provided solely for purposes of illustration and are not to be construed as limiting the scope of the disclosure in any manner.

### EXAMPLE 1

### Preparation of 6-(fluorochroman-2-yl)methanol (Formula VIII)

A round bottom flask is charged with 6-fluorochroman-2-carboxylic acid (100 g) and tetrahydrofuran (1 L) and the mixture is cooled to 0° C. Sodium borohydride (48.19 g) is slowly added at 0-5° C., then a solution of conc. sulfuric acid (33.95 mL) in ether (68 mL) is added by drops at 0-5° C. and the mixture is stirred at 35° C. for 30-45 minutes. Reaction completion is verified using thin layer chromatography (TLC), and the reaction mixture is cooled to 0° C. followed by quenching of the reaction by slow addition of methanol (300 mL). The solvent is distilled completely under reduced pressure, water (500 mL) is added to the residue, and pH of the mixture is adjusted to 7 by addition by drops of sodium hydroxide solution. The mass is extracted with dichloromethane (1000 mL, then 500 mL), followed by washing of the combined organic layer with water (500 mL) and brine solution (500 mL) and drying with sodium sulfate (50 g). The solvent is distilled under reduced pressure to afford the title compound (85 g, 92% yield).

### EXAMPLE 2

### Preparation of 6-fluorochroman-2-carboxaldehyde (Formula VII)

A round bottom flask is charged with 6-(fluorochroman-2-yl)methanol (84 g) and dichloromethane (840 mL), the mixture is stirred at 20° C. for 10 minutes, then sodium bicarbonate solution (116 g in 1160 mL of water) is added and the mass is stirred vigorously for 10 minutes at 20° C. Iodine (234.2 g) and TEMPO (7.2 g) are added, the mixture is stirred at 20-25° C. for ˜24 hours, and reaction completion is verified using TLC. The mass is cooled to 0-5° C. and sodium thiosulfate solution (17.4 g in 170 mL of water) is added and the mixture is stirred for 10 minutes. The organic layer is separated and the aqueous layer is extracted with dichloromethane (168 mL). The organic layers are combined, washed with water (840 mL) and brine solution, and dried with sodium sulfate. The solvent is distilled under reduced pressure below 35° C., to afford the title compound (75 g, 90% yield).

### EXAMPLE 3

### Preparation of 6-fluoro-3, 4-dihydro-2-oxiranyl-2H-1-benzopyran (Formula VI)

A round bottom flask is charged with sodium hydride (17.8 g) in dimethylsulfoxide (876 mL). To the mixture, a solution of trimethylsulfoxonium iodide (89.1 g) in DMSO (714 mL) is slowly added over 20 minutes, and the mixture is stirred for 30 minutes. A solution of 6-fluorochroman-2-carboxaldehyde (73 g) in dichloromethane (150 mL) is added over 20 minutes and the mixture is stirred for 1 hour at 20-25° C. Reaction completion is verified using TLC and the reaction is quenched by adding ice-cold water (730 mL). The mixture is extracted with dichloromethane (730 mL, then 145 mL) and the organic layer is washed with water (2×220 mL), 10% sodium bicarbonate solution (150 mL), and brine solution (150 mL). The organic layer is separated, dried over sodium sulfate and the solvent is distilled under vacuum below 35° C., to afford the title compound (71 g, 90% yield). The isomers are separated by column chromatography with a silica gel support, using ethyl acetate and hexane as an eluant, to afford A-isomer and B-isomer in yields of 45% and 24%, respectively.

### EXAMPLE 4

### Preparation of [R*(S*)]-6-fluoro-3,4-dihydro-[α]-[[(phenylmethyl)-amino]methyl]-2H-1-benzopyran-2-methanol (Formula VA)

A round bottom flask is charged with [R*(S*)]-6-fluoro-3,4-dihydro-2-oxiranyl-2H-1-benzopyran (15 g), isopropyl alcohol (225 mL) and benzyl amine (24.8 g). The mixture is stirred under reflux conditions for 2-3 hours and reaction completion is verified using TLC. The solvent is distilled under vacuum below 50-55° C. Hexane (150 mL) is added to the residue and the mixture is cooled to 0-5° C. The formed solid is filtered and washed with hexane (75 mL), then dried under vacuum at room temperature to afford the title compound in 73% yield. HPLC purity=97.17%.

Alternately, the compound of Formula VB can be prepared with this procedure, starting with [R*(R*)]-6-fluoro-3,4-dihydro-2-oxiranyl-2H-1-benzopyran.

### EXAMPLE 5

### Preparation of [R*S*S*S*]+[R*S*R*R*]-α,α′-[[phenylmethyl)imino]bismethylene]bis[6-fluoro-3,4-dihydro-2H-1-benzopyran-2-methanol] (Formula IV, when P=benzyl))

A round bottom flask is charged with (R*S*)-6-fluoro-3,4-dihydro-α-[[phenylmethyl)amino]methyl]-2H-1-benzopyran-2-methanol (15 g, obtained in Example 4), (R*R*)-6-fluoro-3,4-dihydro-2-oxiranyl-2H-1-benzopyran (12 g), and isopropyl alcohol (225 mL). The mixture is heated to reflux temperature, stirred for 8 hours, and maintained at the same temperature for completion of the reaction (14-15 hours), as verified using TLC. The mixture is cooled to 45-50° C. and solvent is distilled under vacuum below 50-55° C., to afford the title compound in 93.4% yield.

### EXAMPLE 6

### Preparation of [R*S*S*S*]+[R*S*R*R*]-α,α′-iminobismethylene]bis[6-fluoro-3,4-dihydro-2H-1-benzopyran-2-methanol] (Formula III)

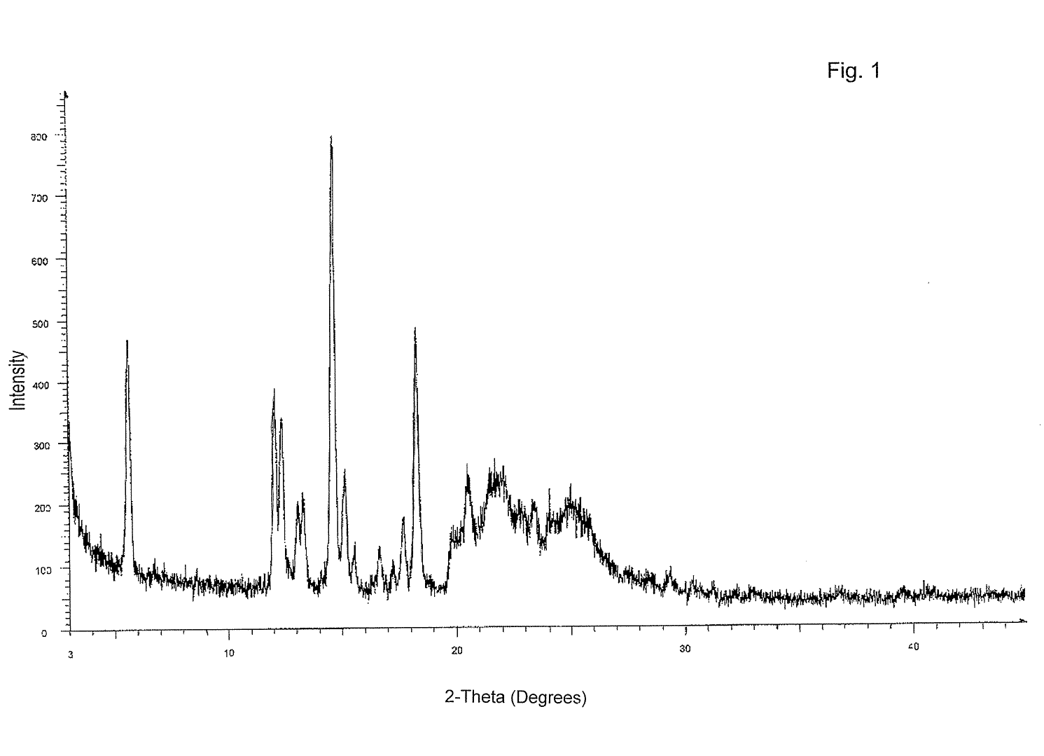

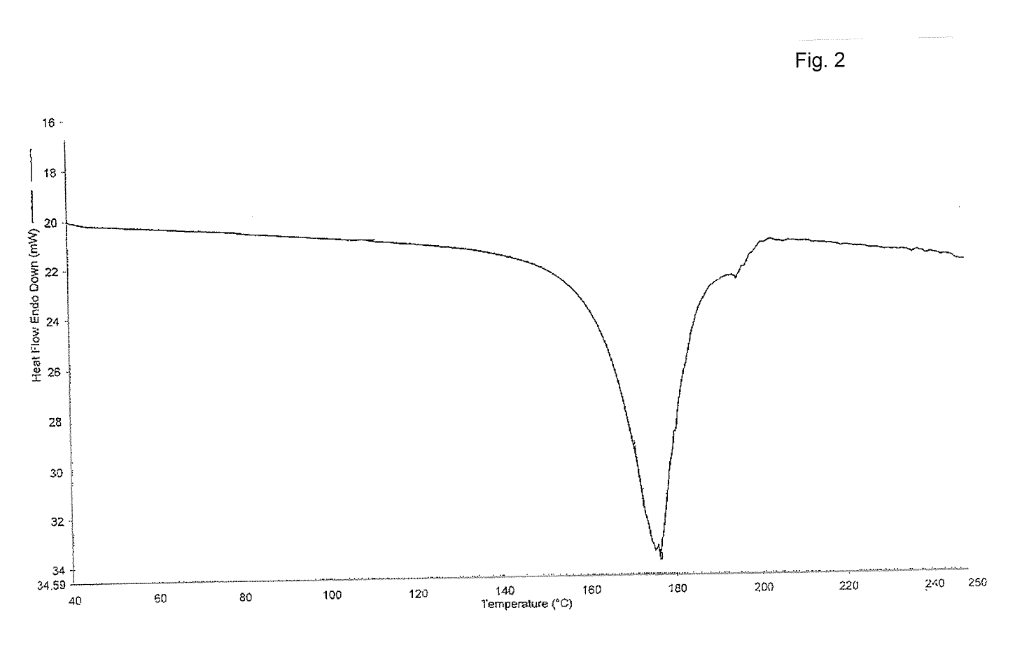

A mixture of [R*S*S*S*]+[R*S*R*R*]-α-α′-[phenylmethyliminobis(methylene)bis[6-fluoro-3,4-dihydro-2H-1-benzopyran-2-methanol] residue (20 g, obtained in Example 5), 10% palladium on charcoal (10 g), ammonium formate (12.6 g), and methanol (200 mL) are stirred under reflux conditions for 2-3 hours. After reaction completion, as verified using TLC, the mixture is filtered and the solid is washed with methanol (40 mL). The filtrate is distilled under vacuum below 50-55° C., to afford the title compound in 91.79% yield. The PXRD pattern is shown as FIG. 1. The DSC curve is shown as FIG. 2.

### EXAMPLE 7

### Preparation of [R*S*S*S*]+[R*S*R*R*]-α,α′-iminobismethylene]bis[6-fluoro-3,4-dihydro-2H-1-benzopyran-2-methanol] hydrochloride (Formula II)

A round bottom flask is charged with [R*S*S*S*]+[R*S*R*R*]-α-α′-[iminobis(methylene)bis[6-fluoro-3,4-dihydro-2H-1-benzopyran-2-methanol] (12 g) and methanol (120 mL). To this mixture, aqueous HCl (4.2 mL, 0.0345 moles) is slowly added at room temperature and the mass is stirred for formation of a thick solid. The solid is filtered and washed with methanol (36 mL), then dried under vacuum at 50° C. to afford the title compound in 38.22% yield.

### EXAMPLE 8

### Preparation of Nebivolol Hydrochloride (Formula I)

A mixture of [R*S*S*S*]+[R*S*R*R*]-α-α′-[iminobis(methylene)bis[6-fluoro-3,4-dihydro-2H-1-benzopyran-2-methanol] hydrochloride salt (4 g, obtained in Example 7) and methanol (120 mL) is heated to reflux temperature and stirred to produce a clear solution. The solvent is distilled to 65-75% of the original solution volume and the solution is cooled to room temperature and stirred for 1-2 hours. The formed solid is filtered, washed with methanol (12 mL) and dried under vacuum at 50° C., to afford the compound (3g, 75% yield) having an (R*S*S*S*):(R*S*R*R*) ratio of 98.93:1.06. The material (2 g) is mixed with methanol (6 mL), ethanol (20 mL), and hexane (20 mL) and stirred for 1-2 hours at room temperature, and then the solid is filtered, washed with hexane (20 mL), dried under vacuum at 50° C. to afford the title compound in 75% yield.

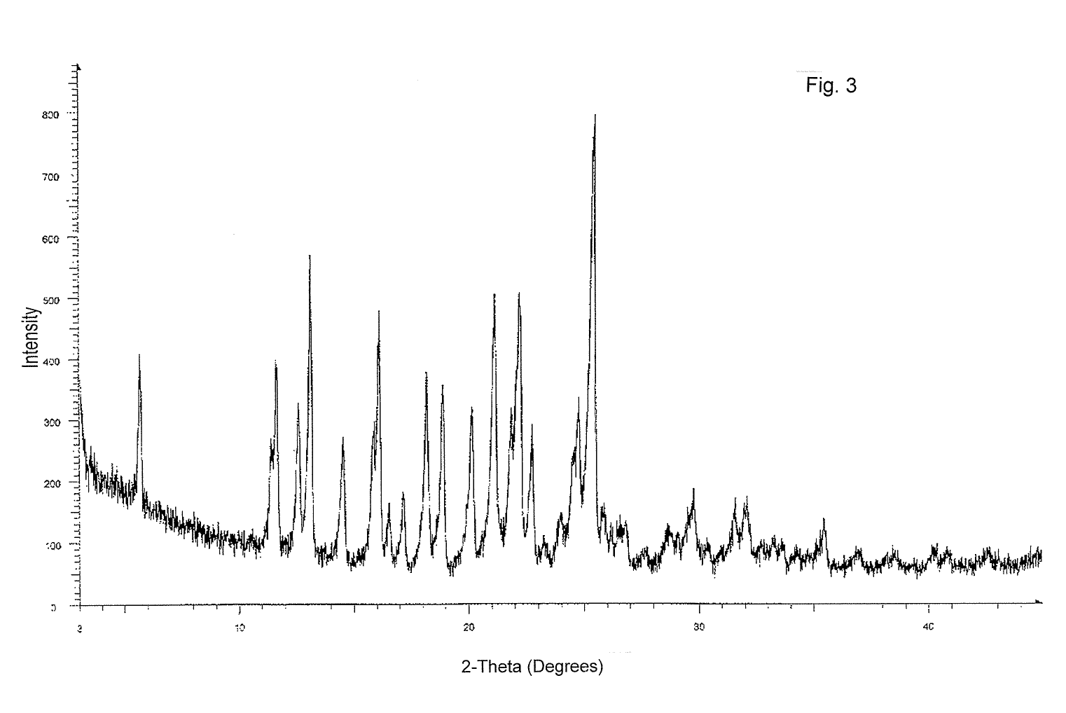

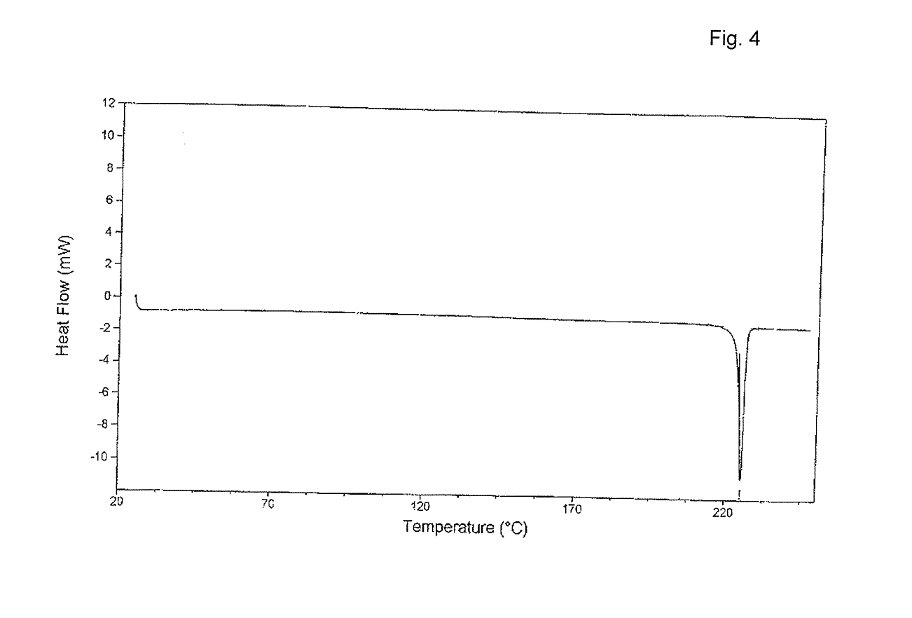

The ratio of (R*S*S*S*) to (R*S*R*R*) is 99.79:0.20. The particle size distribution, determined using a Malvern instrument, is: D₉₀=48.4 μm; D₁₀=3.94 μm; and D₅₀=19.1 μm. Specific surface area (BET)=1.2580 m²/g. The PXRD pattern is shown as FIG. 3. The DSC curve is shown as FIG. 4.

### EXAMPLE 9

### Preparation of (R*S*)-6-fluoro-3,4-dihydro-α-(aminomethyl)-2H-1-benzopyran-2-methanol (Formula VA, when P═H)

A round bottom flask is charged with aqueous ammonia (25%, 1800 mL, 25 moles) and methanol (50 mL). The mixture is cooled to 10-15° C. and a solution of (R*S*)-6-fluoro-2-(oxiran-2-yl)chroman (9 g) in methanol (50 mL) is added at 10-15° C., over 3-4 hours. The mixture is slowly warmed to 25-35° C. and maintained at that temperature for 1-2 hours, and reaction completion is verified using TLC. The mixture is extracted with dichloromethane (2×250 mL), followed by distillation to afford the title compound (8.5 g, 87% yield). HPLC purity=92.97%.

### EXAMPLE 10

### Preparation of [R*S*S*S*]+[R*S*R*R*]-α,α′-iminobismethylene]bis[6-fluoro-3,4-dihydro-2H-1-benzopyran-2-methanol] (Formula III)

A round bottom flask is charged with (R*S*)-6-fluoro-3,4-dihydro-aminomethyl-2H-1-benzopyran-2-methanol (8 g, obtained in Example 9), (R*R*)-6-fluoro-3,4-dihydro-2-oxiranyl-2H-1-benzopyran (8.1 g), and methanol (20 mL). The mixture is heated to reflux temperature and stirred for 10-12 hours, and reaction completion is verified using TLC. The solvent is distilled completely below 50° C., ethanol is added to the residue, and the mixture is stirred for 30-45 minutes at room temperature. The solid is filtered, washed with ethanol (10 mL), and dried under vacuum at 50-55° C., to afford the title compound (4.5 g, 29.3% yield).

### EXAMPLE 11

### Preparation of [R*S*S*S*]+[R*S*R*R*]-α,α′-iminobismethylene]-bis[6-fluoro-3,4-dihydro-2H-1-benzopyran-2-methanol] hydrochloride (Formula II)

A round bottom flask is charged with [R*S*S*S*] +[R*S*R*R1-α-α′-[iminobis(methylene)bis[6-fluoro-3,4-dihydro-2H-1-benzopyran-2-methanol] (4 g) and methanol (40 mL). To this mixture, aqueous HCl (1.4 mL) is slowly added to produce a clear solution. The mixture is stirred at room temperature and thick solid forms. The solid is filtered, washed with methanol (12 mL), and dried under vacuum at 50° C. to afford the title compound (1.8 g, 41.28% yield).

### EXAMPLE 12

### Preparation of Nebivolol Hydrochloride (Formula I)

A mixture of [R*S*S*S*]+[R*S*R*R*]-α-α′-[iminobis(methylene)bis[6-fluoro-3,4-dihydro-2H-1-benzopyran-2-methanol] hydrochloride salt (1.6 g, obtained in Example 11) and methanol (48 mL) is heated to reflux temperature and stirred to produce a clear solution. The solvent is distilled to 65-75% of the initial solution volume and the solution is cooled to room temperature and stirred for 1-2 hours. The solid is filtered, washed with methanol (4.8 mL), and dried under vacuum at 50° C. to afford the compound (1.2 g, 75% yield) having a (R*S*S*S*):(R*S*R*R*) ratio of 97.70:1.172.

To enhance the purity, the material (1 g) is stirred with methanol (1 mL), ethanol (10 mL), and hexane (10 mL) for 1 hour at room temperature, then the solid is filtered, washed with hexane (10 mL), and dried under vacuum at 50° C. to afford the title compound (0.7 g, 70% yield).

### EXAMPLE 13

### Preparation of α,α′-[[(phenylmethyl)imino]bismethylene]bis[6-fluoro-3,4-dihydro-2H-1-benzopyran-2-methanol] (Formula IV′, where P=benzyl)

A round bottom flask is charged with [(R*S*)+(R*R*)]-6-fluoro-2-(oxiran-2-yl)chroman (25.3 g), isopropyl alcohol (350 mL), and benzylamine (6 g). The mixture is stirred under reflux conditions and reaction completion is verified using TLC. The solvent is distilled under vacuum below 50-55° C., to afford the title compound (35 g, ˜100% yield).

### EXAMPLE 14

### Preparation of α,α′-iminobismethylene]bis[6-fluoro-3,4-dihydro-2H-1-benzopyran-2-methanol] (Formula III′)

A mixture of α-α′-[phenylmethyliminobis(methylene)bis[6-fluoro-3,4-dihydro-2H-1-benzopyran-2-methanol] residue (35 g, obtained in Example 13), 10% palladium on charcoal (17.5 g), ammonium formate (22.9 g), and methanol (350 mL) is stirred under reflux conditions for 2-3 hours. Reaction completion is verified using TLC, the mixture is filtered through a Celite bed, and the bed is washed with methanol (70 mL). The filtrate is distilled under vacuum below 50-55° C., to afford the title compound in 56.6% yield.

### EXAMPLE 15

### Preparation of α,α′-iminobismethylene]bis[6-fluoro-3,4-dihydro-2H-1-benzopyran-2-methanol] hydrochloride (Formula II′)

A round bottom flask is charged with α,α′-[iminobis(methylene)bis[6-fluoro-3,4-dihydro-2H-1-benzopyran-2-methanol] (15 g, obtained in Example 14) and methanol (150 mL). To this mixture, aq. HCl (8 g, 0.219 moles) is slowly added at room temperature and the mixture is stirred for thick solid formation at 25-35° C. The solid is filtered, washed with methanol (30 mL), and dried under vacuum at 50° C., to afford the title compound in 20.2% yield.

### EXAMPLE 16

### Preparation of Nebivolol Hydrochloride (Formula I)

A round bottom flask is charged with α-α′-[iminobis(methylene)bis[6-fluoro-3,4-dihydro-2H-1-benzopyran-2-methanol] hydrochloride salt (2 g, obtained in Example 15) and methanol (60 mL), and the mixture is heated to reflux temperature and stirred to produce a clear solution. The solvent is distilled to 65-75% of the initial solution volume, then the mixture is cooled to room temperature and stirred for 1-2 hours. The formed solid is filtered, washed with methanol (10 mL), and dried under vacuum at 50° C. for 3-4 hours, to afford the title compound (0.9 g, 45% yield).

### EXAMPLE 17

### Preparation of 6-(fluorochroman-2-yl)methanol (Formula VIII)

A round bottom flask is charged with 6-fluorochroman-2-carboxylic acid (5 g) and tetrahydrofuran (100 mL) and the mixture is cooled to 0-5° C. Sodium borohydride (1.18 g) is slowly added followed by addition of sodium sulfate (12.7 g) and boric acid (15 mg) at 0-5° C. Then the reaction mixture is stirred at 25-30° C. for 30 minutes. Reaction completion is verified using thin layer chromatography (TLC), and if the reaction is not complete, the reaction mixture is heated at reflux until completion of reaction. The reaction mixture is cooled to 30° C. and filtered, filtrate is distilled, and dichloromethane (40 mL) is charged to the residue. The organic layer is washed with 15% sodium bicarbonate solution (30 mL) and brine solution (30 mL). Subsequently, organic layer is dried over sodium sulfate (5 g) and distilled under vacuum at 40° C. to afford the title compound (3 g, 65% yield).

### EXAMPLE 18

### Preparation of 6-(fluorochroman-2-yl)methanol (Formula VIII)

A round bottom flask is charged with 6-fluorochroman-2-carboxylic acid (5 g) and tetrahydrofuran (100 mL) and the mixture is cooled to 0-5° C. Sodium borohydride (2.4 g) is slowly added followed by addition of sodium sulfate (12.7 g) and 3,4,5-trifluorophenyl boronic acid (44.8 mg) at 0-5° C. Then the reaction mixture is stirred at 25-30° C. for 10 hours. Reaction completion is verified using thin layer chromatography (TLC), and if the reaction is not complete, the reaction mixture is heated at reflux until completion of reaction. The reaction mixture is cooled to 30° C. and filtered, filtrate is distilled, and dichloromethane (40 mL) is charged to the residue. The organic layer is washed with 15% sodium bicarbonate solution (30 mL) and brine solution (30 mL). Subsequently, organic layer is dried over sodium sulfate (5 g) and distilled under vacuum at 40° C. to afford the title compound (1.9 g, 41% yield).

### EXAMPLE 19

### Preparation of [R*(S*)]-6-fluoro-3,4-dihydro-[α]-[[(phenylmethyl)amino]methyl]-2H-1-benzopyran-2-methanol (Formula VA)

A round bottom flask is charged with [R*(S*)]-6-fluoro-3,4-dihydro-2-oxiranyl-2H-1-benzopyran (200 g), and benzyl amine (552 g). The mixture is stirred at 60-65° C. for 2-3 hours and reaction completion is verified using TLC. The reaction mixture is cooled to room temperature, followed by addition of cyclohexane (1200 mL), the mixture is further cooled to 0-5° C., and maintained at the same temperature for about 1 hour. The formed solid is filtered and washed with cyclohexane (1000 mL), then dried under vacuum at 50-55° C. to afford the title compound in ˜78% yield (242.2 g). HPLC purity=97.51%.

Alternately, the compound of Formula VB can be prepared with this procedure, starting with [R*(R*)]-6-fluoro-3,4-dihydro-2-oxiranyl-2H-1-benzopyran.

### EXAMPLE 20

### Preparation of [R*S*S*S*]+[R*S*R*R*]-α,α′-(iminobis methylene)bis(6-fluoro-3,4-dihydro-2H-1-benzopyran-2-methanol) (Formula III)

A round bottom flask is charged with (R*S*)-6-fluoro-3,4-dihydro-aminomethyl-2H-1-benzopyran-2-methanol (1 g), (R*R*)-6-fluoro-3,4-dihydro-2-oxiranyl-2H-1-benzopyran (0.91 g), boric acid (0.308 g), and dimethylsulfoxide (30 mL). The mixture is heated to about 90° C., stirred for 12-16 hours, and reaction completion is verified using thin layer chromatography (TLC).

### EXAMPLE 21

### Preparation of [R*(S*)]-6-fluoro-3,4-dihydro-[α]-[[(phenyl methyl)amino]methyl]-2H-1-benzopyran-2-methanol (Formula VA)

A round bottom flask is charged with [R*(S*)]-6-fluoro-3,4-dihydro-2-oxiranyl-2H-1-benzopyran (20 g) and benzylamine (44.14 g), and the mixture is stirred at room temperature. Boric acid (1.26 g) is added with further stirring at room temperature for 4-6 hours, reaction completion is verified using TLC, and then cyclohexane (100 mL) is added. The solid that forms is collected by filtration, washed with cyclohexane (60 mL), and then dried under vacuum below 55° C. to afford the title compound in about 70% yield. HPLC purity=99.63%.

Alternately, the compound of Formula VB can be prepared using this procedure, starting with [R*(R*)]-6-fluoro-3,4-dihydro-2-oxiranyl-2H-1-benzopyran.

### EXAMPLE 22

### Preparation of [R*(S*)]-6-fluoro-3,4-dihydro-[α]-[[(phenyl methyl)amino]methyl]-2H-1-benzopyran-2-methanol (Formula VA)

A round bottom flask is charged with [R*(S*)]-6-fluoro-3,4-dihydro-2-oxiranyl-2H-1-benzopyran (20 g), benzyl amine (44.14 g), and the mixture is stirred at room temperature. Boric acid (0.62 g) is added with further stirring at room temperature for about 8 hours, reaction completion is verified using TLC, and then cyclohexane (100 mL) is added. The solid that forms is collected by filtration, washed with cyclohexane (160 mL), and then dried under vacuum below 50° C. to afford the title compound in about 68% yield. HPLC purity=97.58%.

Alternately, the compound of Formula VB can be prepared using this procedure, starting with [R*(R*)]-6-fluoro-3,4-dihydro-2-oxiranyl-2H-1-benzopyran.

### EXAMPLE 23

### Preparation of α,α′-(iminobismethylene)bis(6-fluoro-3,4-dihydro-2H-1-benzopyran-2-methanol) (Formula III)

A round bottom flask is charged with (R*S*)-6-fluoro-3,4-dihydro-α-[[phenylmethyl)amino]methyl]-2H-1-benzopyran-2-methanol (20 g), (R*R*)-6-fluoro-3,4-dihydro-2-oxiranyl-2H-1-benzopyran (14.1 g), and methanol (60 mL). The mixture is heated to reflux temperature and maintained at the same temperature until completion of the reaction (18-20 hours), as verified using TLC. The mixture is cooled to 30° C., followed by addition of methanol (225 mL), 10% Pd on carbon (2 g), water (15 mL), and ammonium formate (20.9 g). The mixture is stirred under reflux conditions for about 3 hours (during reflux, an additional 100 mL of methanol is added), and reaction completion is verified using TLC. The mixture is cooled to about 45° C., filtered, and the solid obtained is washed with methanol (40 mL). The filtrate is completely evaporated under vacuum below 45° C. Cyclohexane (100 mL) is added to the residue obtained and the mixture is stirred at room temperature (30° C.) for about 2 hours. The resulting solid is collected by filtration, washed with cyclohexane (50 mL), then dried under vacuum below 50° C. to afford the title compound of enhanced purity (diastereomeric purity about 88%).

### EXAMPLE 24

### Preparation of α,α′-[[(phenylmethyl)imino]bismethylene]bis[6-fluoro-3,4-dihydro-2H-1-benzopyran-2-methanol] of enhanced purity (Formula IIIA, where P=benzyl))

A round bottom flask is charged with (R*S*)-6-fluoro-3,4-dihydro-α-[[phenylmethyl)amino]methyl]-2H-1-benzopyran-2-methanol (20 g), (R*R*)-6-fluoro-3,4-dihydro-2-oxiranyl-2H-1-benzopyran (13.5 g), and isopropyl alcohol (60 mL). The mixture is heated to reflux temperature and stirred at the same temperature for 10-12 hours for completion of the reaction, as verified using TLC. The mixture is cooled to 25-30° C. and stirred for 5-6 hours. The formed solid is filtered and washed with cyclohexane (60 mL), then dried under vacuum at 50-55° C. for 3 hours, to afford the title compound of enhanced isomeric purity. HPLC purity [(R*S*S*S*) to (R*S*R*R*)]=93.98:2.90.

### EXAMPLE 24

### Preparation of α,α′-[[(phenylmethyl)imino]bismethylene]bis[6-fluoro-3,4-dihydro-2H-1-benzopyran-2-methanol] of enhanced purity (Formula IIIA, where P=benzyl))

A round bottom flask is charged with (R*S*)-6-fluoro-3,4-dihydro-α-[[phenylmethyl)amino]methyl]-2H-1-benzopyran-2-methanol (20 g), (R*R*)-6-fluoro-3,4-dihydro-2-oxiranyl-2H-1-benzopyran (13.5 g), and isopropyl alcohol (60 mL). The mixture is heated to reflux temperature and stirred at the same temperature for 10-12 hours, until completion of the reaction, as verified using TLC. The mixture is cooled to 25-30° C., stirred for 5-6 hours. The solid that forms is collected by filtration, washed with cyclohexane (60 mL), and then dried under vacuum at 50-55° C. for 3 hours, to afford the title compound of enhanced isomeric purity. HPLC purity [(R*S*S*S*) to (R*S*R*R*)]=93.98:2.90.

### EXAMPLE 25

### Preparation of α,α′-[[(phenylmethyl)imino]bismethylene]bis[6-fluoro-3,4-dihydro-2H-1-benzopyran-2-methanol] of enhanced purity (Formula IIIA, where P=benzyl))

A round bottom flask is charged with (R*S*)-6-fluoro-3,4-dihydro-α-[[phenylmethyl)amino]methyl]-2H-1-benzopyran-2-methanol (20 g), (R*R*)-6-fluoro-3,4-dihydro-2-oxiranyl-2H-1-benzopyran (13.5 g), and isopropyl alcohol (60 mL). The mixture is heated to reflux temperature and stirred at the same temperature for 10-12 hours, until completion of the reaction, as verified using TLC. The mixture is cooled to 25-30° C. and stirred for 5-6 hours. The solid that forms is collected by filtration, washed with cyclohexane (60 mL), and then dried under vacuum at 50-55° C. for 3 hours, to afford the title compound of enhanced isomeric purity. HPLC purity [(R*S*S*S*) to (R*S*R*R*)]=94.82:2.42.

### EXAMPLE 26

### Preparation of α,α′-iminobismethylene]bis[6-fluoro-3,4-dihydro-2H-1-benzopyran-2-methanol] (Formula IIA) of enhanced purity

A mixture of [R*S*S*S*]+[R*S*R*R*]-α-α′-[phenylmethyliminobis (methylene)bis[6-fluoro-3,4-dihydro-2H-1-benzopyran-2 -methanol] (20 g), methanol (400 mL), water (20 mL), 10% palladium on charcoal (1.60 g), and ammonium formate (7.62 g) is stirred under reflux conditions for 2-3 hours. After reaction completion, as verified using TLC, the mixture is filtered at 50-55° C. and the collected solid is washed with methanol (100 mL). The filtrate is completely evaporated under vacuum below 50-55° C. and cyclohexane (100 mL) is added to the residue. The mixture is stirred at room temperature (30° C.) for about 1 hour. The solid that forms is collected by filtration, washed with cyclohexane (50 mL), and then dried under vacuum at 50-55° C. for 4-5 hours, to afford the title compound in 94.24% yield. HPLC purity (R*S*S*S*)=98.66%.

### EXAMPLE 27

### Preparation of α,α′-[[(phenylmethyl)imino]bismethylene]bis[6-fluoro-3,4-dihydro-2H-1-benzopyran-2-methanol] of enhanced purity (Formula IIIA, where P=benzyl))

A round bottom flask is charged with (R*S*)-6-fluoro-3,4-dihydro-α-[[phenylmethyl)amino]methyl]-2H-1-benzopyran-2-methanol (20 g), (R*R*)-6-fluoro-3,4-dihydro-2-oxiranyl-2H-1-benzopyran (14.7 g), and methanol (60 mL). The mixture is heated to reflux temperature, stirred at the same temperature for 10-12 hours, until completion of the reaction, as verified using TLC. The mixture is cooled to room temperature (25-30° C.) and stirred until solid forms (about 2 hours). The mixture is further cooled to 0-5° C., stirred until a thick solid forms, then cyclohexane (100 mL) is added with stirring. The resulting solid is collected by filtration, washed with cyclohexane (40 mL), and then dried under vacuum at 50-55° C. for about 3 hours, to afford the title compound of enhanced isomeric purity. HPLC purity (R*S*S*S*)=93.68%.

### EXAMPLE 28

### Preparation of α,α′-[[(phenylmethyl)imino]bismethylene] bis[6-fluoro-3,4-dihydro-2H-1-benzopyran-2-methanol] of enhanced purity (Formula IIIA, where P=benzyl))

A round bottom flask is charged with (R*S*)-6-fluoro-3,4-dihydro-α-[[phenylmethyl)amino]methyl]-2H-1-benzopyran-2-methanol (20 g), (R*R*)-6-fluoro-3,4-dihydro-2-oxiranyl-2H-1-benzopyran (14.7 g), and isopropyl alcohol (100 mL). The mixture is heated to reflux temperature, stirred at the same temperature for 12-14 hours, until completion of the reaction, as verified using TLC. The mixture is cooled to room temperature (25-30° C.) and stirred until solid forms (about 3-4 hours). The solid is isolated by filtration, washed with isopropyl alcohol (50 mL), and then dried under vacuum at 50-55° C., to afford the title compound of enhanced isomeric purity. HPLC purity (R*S*S*S*)=93.89%.

### EXAMPLE 29

### Preparation of α,α′-[[(phenylmethyl)imino]bismethylene]bis [6-fluoro-3,4-dihydro-2H-1-benzopyran-2-methanol] of enhanced purity (Formula IIIA, where P=benzyl)).

A round bottom flask is charged with (R*S*)-6-fluoro-3,4-dihydro-α-[[phenylmethyl)amino]methyl]-2H-1-benzopyran-2-methanol (20 g), (R*R*)-6-fluoro-3,4-dihydro-2-oxiranyl-2H-1-benzopyran (14.17 g), and isopropyl alcohol (100 mL). The mixture is heated to reflux temperature and stirred at the same temperature for 12-14 hours, until completion of the reaction, as verified using TLC. The mixture is cooled to room temperature (25-30° C.) as it becomes hazy. The mixture is further cooled to 4-10° C. with stirring at that temperature for thick solid formation (about 3-4 hours). The solid is isolated by filtration, washed with chilled isopropyl alcohol (50 mL), and then dried under vacuum at 50-55° C., to afford the title compound.

### EXAMPLE 30

### Preparation of α,α′-[[(phenylmethyl)imino]bismethylene]bis [6-fluoro-3,4-dihydro-2H-1-benzopyran-2-methanol] of enhanced purity (Formula IIIA, where P=benzyl))

A round bottom flask is charged with (R*S*)-6-fluoro-3,4-dihydro-α-[[phenylmethyl)amino]methyl]-2H-1-benzopyran-2-methanol (20 g), (R*R*)-6-fluoro-3,4-dihydro-2-oxiranyl-2H-1-benzopyran (14.7 g), and methanol (100 mL). The mixture is heated to reflux temperature and stirred at the same temperature for 12-14 hours, until completion of the reaction, as verified using TLC. The mixture is cooled to 4-10° C. and stirred at that temperature for thick solid formation (about 3-4 hours). The solid is isolated by filtration, washed with methanol (40 mL), and then dried under vacuum at 50-55° C., to afford the title compound having HPLC purity (R*S*S*S*)=91.46%.

### EXAMPLE 31

### Preparation of α,α′-[[(phenylmethyl)imino]bis methylene]bis [6-fluoro-3,4-dihydro-2H-1-benzopyran-2-methanol] of enhanced purity (Formula IIIA, where P=benzyl))

A round bottom flask is charged with (R*S*)-6-fluoro-3,4-dihydro-α-[[phenylmethyl)amino]methyl]-2H-1-benzopyran-2-methanol (20 g), (R*R*)-6-fluoro-3,4-dihydro-2-oxiranyl-2H-1-benzopyran (14.7 g), and methanol (100 mL). The mixture is heated to reflux temperature and stirred at the same temperature for 12-14 hours, until completion of the reaction, as verified using TLC. The mixture is cooled to room temperature (25-35° C.) and stirred at that temperature for thick solid formation (about 3-4 hours). The resulting solid is collected by filtration, washed with methanol (40 mL), and then dried under vacuum at 50-55° C. for about 3 hours, to afford the title compound having HPLC purity (R*S*S*S*)=96.56%.

### EXAMPLE 32

### Preparation of α,α′-[[(phenylmethyl)imino]bis methylene]bis[6-fluoro-3,4-dihydro-2H-1-benzopyran-2-methanol] of enhanced purity (Formula IIIA, where P=benzyl))

A round bottom flask is charged with (R*S*)-6-fluoro-3,4-dihydro-α-[[phenylmethyl)amino]methyl]-2H-1-benzopyran-2-methanol (20 g), (R*R*)-6-fluoro-3,4-dihydro-2-oxiranyl-2H-1-benzopyran (14.7 g), and methanol (200 mL). The mixture is heated to reflux temperature and stirred at the same temperature for 22-24 hours, until completion of the reaction, as verified using TLC. The mixture is cooled to room temperature and evaporated under vacuum below 55° C., followed by drying under vacuum to afford a residue [R*S*S*S*:R*S*R*R*=49.9:44.0]. Methanol (100 mL) is added to the residue and the mixture is heated to about 60-65° C. for dissolution. The solution is stirred at room temperature (25-35° C.) for about 1.5-2 hours for solid formation. The collected solid has 90.99% (R*S*S*S*) purity by HPLC.

### EXAMPLE 33

### Preparation of Substantially Pure α,α′-(imino dimethylene)bis(6-fluoro-2-chromanmethanol)

A round bottom flask is charged with (R*S*)-6-fluoro-3,4-dihydro-α-[[phenylmethyl)amino]methyl]-2H-1-benzopyran-2-methanol (10 g) and (R*R*)-6-fluoro-3,4-dihydro-2-oxiranyl-2H-1-benzopyran (7.04 g). The mixture is heated to 100-110° C. and stirred at this temperature until completion of the reaction (6-8 hours), as verified using TLC. The mixture is cooled to 45-50° C. and methanol (50 mL) is slowly added at 45-50° C. 10% Palladium on carbon (3 g) and water (5 mL) are added to the mixture followed by addition of ammonium formate (12.54 g). Additional methanol (100 mL) is added to the mixture. The mixture is heated to 60-65° C. and maintained at this temperature until completion of the reaction (about 2 hours), as verified using TLC. The mixture is cooled to 45° C., filtered, and the collected solid is washed with methanol (50 mL). The filtrate is completely evaporated under vacuum below 45-50° C. The residue is dried under vacuum at 50-55° C. for about 2 hours to afford the title compound, having HPLC purity (R*S*S*S*)=82.78%.

### EXAMPLE 34

### Preparation of substantially pure α,α′-(iminodimethylene)bis [6-fluoro-2-chromanmethanol

A round bottom flask is charged with crude α,α′-[[(phenylmethyl)imino]bismethylene]bis[6-fluoro-3,4-dihydro-2H-1-benzopyran-2-methanol] (10 g) and methanol (500 mL), followed by addition of 10% palladium on carbon (3 g). Hydrogen gas pressure of 2-3 kg/cm² is applied and the mixture is stirred at the same pressure for about 3 hours, until completion of the reaction, as verified using TLC. The mixture filtered through a Hyflow (flux calcined diatomaceous earth) bed and the bed is washed with methanol (40 mL). The filtrate is completely evaporated under vacuum below 45-50° C. to afford the title compound having HPLC purity (R*S*S*S*)=84.28%.

### EXAMPLE 35

### Preparation of Nebivolol Hydrochloride

A mixture of substantially pure α,α′-[[(phenylmethyl)imino]bismethylene]bis[6-fluoro-3,4-dihydro-2H-1-benzopyran-2-methanol] (25 g), 10% palladium on charcoal (1.25 g), ammonium formate (6.3 g), and methanol (475 mL) is stirred under reflux conditions for 2-3 hours. Reaction completion is verified using TLC, then the mixture is cooled, filtered through a Celite™ bed under vacuum at 50-55° C., and the bed is washed with methanol (125 mL). Dry hydrogen chloride gas is passed through the filtrate until the pH is 2 or less. The mixture is stirred at room temperature for 2-3 hours, then the solid that forms is collected by filtration, washed with methanol (50 mL), and dried under vacuum at 50-55° C. to afford the title compound in 74.98% yield.

### EXAMPLE 36

### Preparation of Nebivolol Hydrochloride

A mixture of substantially pure α,α′-[[(phenylmethyl)imino]bismethylene]bis[6-fluoro-3,4-dihydro-2H-1-benzopyran-2-methanol] (25 g), 10% palladium on charcoal (1.25 g), ammonium formate (6.3 g), and methanol (475 mL) is stirred under reflux conditions for 2-3 hours. Reaction completion is verified using TLC, then the mixture is cooled, filtered through a Celite™ bed under vacuum at 50-55° C., and the bed is washed with methanol (125 mL). Aqueous hydrochloric acid (6.6 mL) is added to the filtrate and the mixture is stirred at room temperature for 2-3 hours. Then 70-80% of the solvent is evaporated under vacuum at 50-55° C. and the mixture is stirred at 0-10° C. for 30 minutes. The formed solid is collected by filtration, washed with methanol (125 mL), and dried under vacuum at 50-55° C. to afford the title compound in 79.9% yield.

### EXAMPLE 37

### Purification of Nebivolol Hydrochloride

A round bottomed flask is charged with the compound obtained in Example 35 (10 g) and methanol (150 mL). The mixture is heated at reflux to produce a clear solution, followed by addition of methanol (50 mL). The mixture is completely evaporated under vacuum to afford a residue. To the residue, ethyl acetate (50 mL) is added and the mixture is stirred at room temperature for about 2 hours. The solid is collected by filtration, washed with ethyl acetate (20 mL), and then dried under vacuum at 50-55° C. The dried solid is mixed with methanol (200 mL) and the mixture is heated at reflux, followed by addition of methanol in portions (2×50 mL) under reflux conditions. The mixture is stirred for about 1 hour to produce a clear solution, and then the solvent is completely evaporated. Ethyl acetate (70 mL) is added to the residue and the mixture is stirred at room temperature for about 1-2 hours. The solid is collected by filtration, washed with ethyl acetate (30 mL), and then dried under vacuum at 50-55° C. to afford the title compound in 94% yield.

### EXAMPLE 38

### Preparation of [R*S*S*S*]+[R*S*R*R*]-α,α′-iminobis methylene]bis[6-fluoro-3,4-dihydro-2H-1-benzopyran-2-methanol] (Formula III)

A round bottom flask is charged with (R*S*)-6-fluoro-3,4-dihydro-aminomethyl-2H-1-benzopyran-2-methanol (10 g), boric acid (3.08 g), (R*R*)-6-fluoro-3,4-dihydro-2-oxiranyl-2H-1-benzopyran (9.19 g), and dimethylsulfoxide (300 mL). The mixture is heated to 90° C., stirred for 14-16 hours, and reaction completion is verified using HPLC. The mixture is poured into a beaker and water (400 mL) is added, followed by addition of ethyl acetate (500 mL). The organic layer is separated and the aqueous layer is extracted with ethyl acetate (2×500 mL). The combined organic layers are completely evaporated under vacuum. Methanol (80 mL) is added to the residue and the mixture is stirred for 15-30 minutes at room temperature. The solid is collected by filtration, and dried to obtain the product.

### EXAMPLE 39

### Preparation of Nebivolol Hydrochloride

A round bottom flask is charged with α,α′-[iminobis(methylene)bis[6-fluoro-3,4-dihydro-2H-1-benzopyran-2-methanol] (10 g) and methanol (150 mL). Aqueous HCl (11N, 3.3 mL) is slowly added at room temperature and the mixture is stirred for thick solid formation at 25-35° C. The solid is collected by filtration, washed with ethyl acetate (20 mL), and dried under vacuum at 50° C., to afford the title compound in 69% yield.

### EXAMPLE 40

### Preparation of [R*(S*)]-6-fluoro-3,4-dihydro-[α]-[[(phenylmethyl)amino] methyl]-2H-1-benzopyran-2-methanol (Formula VA)

A round bottom flask is charged with [R*(S*)]-6-fluoro-3,4-dihydro-2-oxiranyl-2H-1-benzopyran (20 g) and benzyl amine (44.14 g), the mixture is heated to 60-65° C. and stirred at same temperature for about 2 hours, and reaction completion is verified using TLC. The mixture is cooled to room temperature, followed by addition of cyclohexane (200 mL) and subsequent cooling and maintenance at 0-5° C. for about 1 hour. The formed solid is collected by filtration and washed with cyclohexane (60 mL), then dried under vacuum below 55° C. to afford the title compound in about 81.2% yield. HPLC purity=99.37%.

Alternately, the compound of Formula VB can be prepared using this procedure, starting with [R*(R*)]-6-fluoro-3,4-dihydro-2-oxiranyl-2H-1-benzopyran.

### EXAMPLE 41

### Preparation of α,α′-[[(phenylmethyl)imino]bis methylene]bis[6-fluoro-3,4-dihydro-2H-1-benzopyran-2-methanol] of enhanced purity (Formula IIIA, where P=benzyl))

A round bottom flask is charged with (R*S*)-6-fluoro-3,4-dihydro-α-[[phenylmethyl)amino]methyl]-2H-1-benzopyran-2-methanol (Formula VA, 20 g), (R*R*)-6-fluoro-3,4-dihydro-2-oxiranyl-2H-1-benzopyran (Formula VIB, 13.5 g), and isopropyl alcohol (60 mL). The mixture is heated to reflux temperature and stirred at the same temperature for 10-12 hours for completion of the reaction, as verified using TLC. The mixture is cooled to 25-30° C. and stirred for 5-6 hours. The formed solid is collected by filtration and washed with cyclohexane (60 mL), then dried under vacuum at 50-55° C. for 3 hours to afford the title compound of enhanced isomeric purity. HPLC purity [(R*S*S*S*):(R*S*R*R*)]=93.98:2.90.

### EXAMPLE 42

### Preparation of α,α′-[[(phenylmethyl)imino]bis methylene] bis[6-fluoro-3,4-dihydro-2H-1-benzopyran-2-methanol] of enhanced purity (Formula IV, where P=benzyl))

A round bottom flask is charged with (R*S*)-6-fluoro-3,4-dihydro-α-[[phenylmethyl)amino]methyl]-2H-1-benzopyran-2-methanol (Formula VA, 20 g), (R*R*)-6-fluoro-3,4-dihydro-2-oxiranyl-2H-1-benzopyran (Formula VIB, 14.84 g), and methanol (100 mL). The mixture is heated to reflux temperature and stirred at the same temperature for 16-20 hours for completion of the reaction, as verified using TLC. The mixture is cooled to 45-50° C. and seeded with the title compound and further maintained for 30 minutes-2 hours. Then, the reaction mixture is cooled to 25-30° C. and stirred at same for another 30 minutes-2 hours. Subsequently, it is cooled to 5-10° C. and stirred at same for 2-3 hours. The solid formed is collected by filtration and washed with chilled methanol (20 mL, 40 mL) and dried under vacuum at 50-55° C. for 6-10 hours to afford the title compound. The solid has 98.20% (R*S*S*S*) purity by HPLC.

### EXAMPLE 43

### Preparation of Nebivolol Hydrochloride

A mixture of substantially pure α,α′-[[(phenylmethyl)imino]bismethylene]bis[6-fluoro-3,4-dihydro-2H-1-benzopyran-2-methanol] (25 g), 10% palladium on charcoal (1.25 g), ammonium formate (6.3 g), and methanol (475 mL) is stirred under reflux conditions for 2-3 hours. Reaction completion is verified using TLC, then the mixture is cooled and filtered through a Celite™ bed under vacuum at 50-55° C., and the bed is washed with methanol (125 mL). Aqueous hydrochloric acid (6.6 mL) is added to the filtrate and the mixture is stirred at room temperature for 2-3 hours. Then 70-80% of the solvent is evaporated under vacuum at 50-55° C. and the mixture is stirred at 0-10° C. for 30 minutes. The formed solid is collected by filtration, washed with methanol (125 mL), and dried under vacuum at 50-55° C. to afford the title compound in 79.9% yield.

### EXAMPLE 44

### Preparation of Nebivolol Hydrochloride with 1.2% Catalyst

A round bottom flask is charged with α,α′-[[(phenylmethyl)imino]bis methylene]bis[6-fluoro-3,4-dihydro-2H-1-benzopyran-2-methanol] (10 g), methanol (600 mL) and 10% palladium on carbon (0.12 g) in water (0.65 mL). A hydrogen gas pressure of about 5 kg/cm² is applied and the mixture is heated to about 50° C. and stirred at the same temperature and pressure for about 1.5 hours until completion of the reaction, as verified using TLC. The mixture is cooled to about 45° C., filtered through a Hyflow (flux calcined diatomaceous earth) bed, and the bed is washed with methanol (20 mL). Concentrated hydrochloric acid (50 mL) is added to the filtrate at 25-30° C. over 30 minutes and the mixture is stirred at room temperature for 3-4 hours. The solid obtained is collected by filtration, washed with methanol (10 mL then 20 mL), and dried under vacuum at 50-55° C. for 4-5 hours to afford the title compound in 63.90% yield. HPLC purity=99.47%, “de-F” nebivolol=0.06%.

### EXAMPLE 45

### Preparation of Nebivolol Hydrochloride with 1.5% Catalyst

A round bottom flask is charged with α,α′-[[(phenylmethyl)imino]bis methylene]bis[6-fluoro-3,4-dihydro-2H-1-benzopyran-2-methanol] (8 g), methanol (480 mL) and 10% palladium on carbon (0.12 g) in water (0.6 mL). A hydrogen gas pressure of about 2 kg/cm² is applied and reaction mixture is heated to 50° C. and then the mixture is stirred at the same temperature and pressure for about 1.5 hours for completion of the reaction, as verified using TLC. The mixture is cooled to about 45° C. and filtered through a Hyflow (flux calcined diatomaceous earth) bed and the bed is washed with methanol (16 mL). Concentrated hydrochloric acid (40 mL) is added to the filtrate at 35-40° C. and the mixture is stirred at room temperature for 2-3 hours. The solid obtained is collected by filtration, washed with methanol (8 and 16 mL), and dried under vacuum at 50° C. for 3-4 hours to afford the title compound in 67.32% yield. HPLC purity =99.32%, “de-F” nebivolol=0.05%.

### EXAMPLE 46

### Preparation of Nebivolol Hydrochloride with 2% Catalyst

A round bottom flask is charged with α,α′-[[(phenylmethyl)imino]bis methylene]bis[6-fluoro-3,4-dihydro-2H-1-benzopyran-2-methanol] (24 g), methanol (1200 mL) and 10% palladium on carbon (0.48 g) in water (2.4 mL). A hydrogen gas pressure of about 1 kg/cm² is applied, the reaction mixture is heated to 55-60° C., and the mixture is stirred at the same temperature and pressure for about 1.5 hours for completion of the reaction, as verified using TLC. The mixture is cooled to about 45-50° C., filtered through a Hyflow (flux calcined diatomaceous earth) bed, and the bed is washed with methanol (48 mL). Concentrated hydrochloric acid (120 mL) is added to the filtrate at 25-30° C. over 10-20 minutes and the mixture is stirred at room temperature for 3-4 hours. The solid obtained is collected by filtration, washed with methanol (24 then 48 mL), and dried under vacuum at 50-55° C. for 4-6 hours to afford the title compound in 71.96% yield. HPLC purity=99.38%, “de-mono-F” nebivolol=0.01%.

### EXAMPLE 47

### Preparation of Nebivolol Hydrochloride with 5% Catalyst

A round bottom flask is charged with α,α′-[[(phenylmethyl)imino]bis methylene]bis[6-fluoro-3,4-dihydro-2H-1-benzopyran-2-methanol] (16 g), methanol (960 mL) and 10% palladium on carbon (0.8 g) in water (4 mL). A hydrogen gas pressure of about 2 kg/cm² is applied and reaction mixture is heated to about 50° C., then the mixture is stirred at the same temperature and pressure for about 1.5 hours for completion of the reaction, as verified using TLC. The mixture is cooled to about 45° C. and filtered through a Hyflow (flux calcined diatomaceous earth) bed and the bed is washed with methanol (32 mL). Concentrated hydrochloric acid (80 mL) is added to the filtrate at room temperature and the mixture is stirred for 2-3 hours at the same temperature. The solid obtained is collected by filtration, washed with methanol (16 then 32 mL), and dried under vacuum at 50° C. for 4-5 hours to afford the title compound in 61.62% yield. HPLC purity=99.52%, “de-mono-F” nebivolol=0.11%.

### EXAMPLE 48

### Preparation of Nebivolol Hydrochloride with 10% Catalyst

A round bottom flask is charged with α,α′-[[(phenylmethyl)imino]bis methylene]bis[6-fluoro-3,4-dihydro-2H-1-benzopyran-2-methanol] (8 g), methanol (480 mL), and 10% palladium on carbon (0.8 g) in water (4 mL). A hydrogen gas pressure of about 2 kg/cm² is applied and reaction mixture is heated to about 50° C., then the mixture is stirred at the same temperature and pressure for about 1 hour for completion of the reaction, as verified using TLC. The mixture is cooled to about 40-45° C. and filtered through a Hyflow (flux calcined diatomaceous earth) bed and the bed is washed with methanol (16 mL). Concentrated hydrochloric acid (40 mL) is added to the filtrate at room temperature and the mixture is stirred for about 3 hours at the same temperature. The solid obtained is collected by filtration, washed with methanol (8 then 16 mL), and dried under vacuum at 50° C. for 3-4 hours to afford the title compound. HPLC purity=99.13%, “de-mono-F” nebivolol=0.45%.

### EXAMPLE 49

### Preparation of Nebivolol Hydrochloride by CTH Method with 5% Catalyst

A round bottom flask is charged with α,α′-[[(phenylmethyl)imino]bis methylene]bis[6-fluoro-3,4-dihydro-2H-1-benzopyran-2-methanol] (5 g), methanol (250 mL), 10% palladium on charcoal (0.25 g) in water (1.25 mL), and ammonium formate (1.26 g) and is stirred under reflux conditions for about 1 hour. Reaction completion is verified using TLC, then the mixture is cooled to 40-50° C., filtered through a Hyflow bed, and the bed is washed with methanol (10 mL). Dry hydrogen chloride gas is passed through the filtrate until the pH is 2 or less. The mixture is stirred at room temperature for 2-3 hours and the formed solid is collected by filtration and washed with methanol (10 mL). The solid is dried under vacuum at below 50° C. to afford the title compound in 76.4% yield. HPLC purity=98.94%, “de-mono-F” nebivolol=0.36%.

### EXAMPLE 50

### Preparation of Nebivolol Hydrochloride by CTH Method with 5% Ccatalyst

A round bottom flask is charged with α,α′-[[(phenylmethyl) imino]bismethylene]bis[6-fluoro-3,4-dihydro-2H-1-benzopyran-2-methanol] (5 g), methanol (300 mL), 10% palladium on charcoal (0.25 g) in water (0.375 mL), and formic acid (1.4 g) and is stirred under reflux conditions for about 3 hours. Reaction completion is verified using TLC, then the mixture is cooled to 40-50° C., filtered through a Celite™ bed, and the bed is washed with methanol (10 mL). The filtrate is subjected to evaporation under vacuum at 50-55° C. and further dried under vacuum for 2-3 hours to afford the title compound of 97.85% purity.

### EXAMPLE 51

### Preparation of Nebivolol by CTH Method with 15% Catalyst

A round bottom flask is charged with α,α′-[[(phenylmethyl)imino]bis methylene]bis[6-fluoro-3,4-dihydro-2H-1-benzopyran-2-methanol] (5 g), methanol (250 mL), 10% palladium on charcoal (0.75 g) in water (1.25 mL), and ammonium formate (1.26 g) and is stirred at 40-45° C. for completion of reaction, verified using TLC. The mixture is filtered through a Hyflow bed and the bed is washed with methanol (10 mL). The filtrate is subjected to evaporation under vacuum below 50° C. and further dried under vacuum for 2-3 hours to afford the title compound in 95.44% yield. HPLC purity=95.52%, “de-mono-F” nebivolol=0.63%.

### EXAMPLE 52

### Preparation of Nebivolol Hydrochloride with Small Particle Sizes

Nebivolol hydrochloride (10 g) is charged into a dry round bottom flask containing methanol (400 mL), heated to reflux, and stirred for dissolution. The solution is cooled to 45° C., then filtered to remove particles, and subsequently poured into methyl t-butyl ether (700 mL), maintained at 20° C., over 75 minutes at a rate of 5.3 mL/minute. After complete addition, the mixture is stirred at 20° C. for about 2 hours. The solid is collected by filtration and dried under vacuum at 50° C. for about 12 hours. The obtained fine compound (6.5 g) has a particle size distribution with D₉₀=15.180 μm, D₁₀=1.753 μm, and D₅₀=4.571 μm.

### EXAMPLE 53

### Preparation of Nebivolol Hydrochloride with Small Particle Sizes

Nebivolol hydrochloride (10 g) is charged into a dry round bottom flask containing methanol (400 mL), heated to reflux, and stirred for dissolution. The solution is cooled to 45° C., then filtered to remove particles, and subsequently poured into methyl t-butyl ether (700 mL), maintained at 20° C., over about 75 minutes at a rate of 5.55 mL/minute. After complete addition, the mixture is stirred at 20° C. for about 2 hours. The solid is collected by filtration, washed with methyl t-butyl ether (20 mL,) and dried under vacuum at 50° C. for about 12 hours. The obtained fine compound (7.7 g) has a particle size distribution of D₉₀=11.72 μm, D₁₀=1.87 μm, and D₅₀=4.46 μm.

### EXAMPLE 54

### Preparation of Nebivolol Hydrochloride with Small Particle Sizes

Nebivolol hydrochloride (3 g) is charged into a dry round bottom flask containing methanol (120 mL), heated to reflux, and stirred for dissolution. The solution is cooled to room temperature, then filtered to remove particles, and subsequently poured into methyl t-butyl ether (240 mL), maintained at 15-20° C., over about 75 minutes at a rate of 5.55 mL/minute. After complete addition, the mixture is stirred at 15-20° C. for about 1.5 hours. The solid is collected by filtration and dried under vacuum at 50° C. for about 10-12 hours. The obtained fine compound (2.4 g) has a particle size distribution with D₉₀=7.80 μm, D₁₀=1.314 μm, and D₅₀=3.363 μm.

### EXAMPLE 55

### Preparation of Nebivolol Hydrochloride with Small Particle Sizes

Nebivolol hydrochloride (6 g) is charged into a dry round bottom flask containing methanol (240 mL), heated to reflux, and stirred for dissolution. The solution is cooled to room temperature, then filtered to remove particles, and subsequently poured into ethyl acetate (600 mL), which is precooled to 0-5° C., over about 10-15 minutes. After complete addition, the mixture is stirred at 0-5° C. for about 1 hour. The solid is collected by filtration and dried. The obtained fine compound (2.9 g) has a particle size distribution with D₉₀=25.76 μm, D₁₀=2.37 μm, and D₅₀=6.29 μm.

### EXAMPLE 56

### Preparation of Nebivolol Hydrochloride with Small Particle Sizes

Nebivolol hydrochloride (6 g) is charged into a dry round bottom flask containing methanol (240 mL), heated to reflux, and stirred for dissolution. The solution is cooled to room temperature, then filtered to remove particles, and subsequently poured into diethyl ether (600 mL) at room temperature over about 10-15 minutes. After complete addition, the mixture is stirred at room temperature for about 30 minutes. The solid is collected by filtration and dried under vacuum at 60° C. for about 5 hours. The obtained fine compound (5.1 g) has a particle size distribution with D₉₀=10.75 μm, D₁₀=1.23 μm, and D₅₀=3.17 μm. Specific surface area is 3.68 m²/g.

### EXAMPLE 57

### Preparation of [S*(R*)]-6-fluoro-3,4-dihydro-[α]-[[(phenylmethyl)amino]methyl]-2H-1-benzopyran-2-methanol (Formula VA)

A reactor is charged with [S*(R*)]-6-fluoro-3,4-dihydro-2-oxiranyl-2H-1-benzopyran (14 Kg) and benzylamine (30.94 Kg), the mixture is heated to 60-65° C., and stirred at same temperature for about 3 hours, and reaction completion is verified using TLC. The mixture is cooled to 25-35° C., followed by addition of cyclohexane (140 L), subsequent cooling, and maintenance at 5-10° C. for about 2-3 hours. The formed solid is collected by filtration, washed with pre-cooled cyclohexane (42 L then 28 L), and then dried under vacuum below 55° C. to afford the title compound in about 76% yield.

Alternately, the compound of Formula VB can be prepared using this procedure, starting with [R*(R*)]-6-fluoro-3,4-dihydro-2-oxiranyl-2H-1-benzopyran.

### EXAMPLE 58

### Preparation of α,α′-[[(phenylmethyl)imino]bis methylene]bis[6-fluoro-3,4-dihydro-2H-1-benzopyran-2-methanol] of enhanced purity (Formula IV, where P=benzyl))

A reactor is charged with (S*R*)-6-fluoro-3,4-dihydro-α-[[phenylmethyl)amino]methyl]-2H-1-benzopyran-2-methanol (15 Kg), (R*R*)-6-fluoro-3,4-dihydro-2-oxiranyl-2H-1-benzopyran (11.13 Kg), and methanol (79 L). The mixture is heated to reflux temperature and stirred at the same temperature for about 18 hours for completion of the reaction, as verified using TLC. The mixture is cooled to 45-50° C., seeded with the title compound (75 g), and further maintained at the same temperature for 1-2 hours. Then, the mixture is cooled to 25-30° C. and stirred at the same temperature for another 1-2 hours. Subsequently, the reaction mixture is cooled to 5-10° C. and stirred at same temperature for about 3 hours. The formed solid is collected by filtration, washed with precooled methanol (15 L, 30 L), and dried under vacuum at 50-55° C. to afford the title compound.

### EXAMPLE 59

### Preparation of Nebivolol Hydrochloride

A reactor is charged with α,α′-[[(phenylmethyl)imino]bismethylene]bis[6-fluoro-3,4-dihydro-2H-1-benzopyran-2-methanol] (8.50 Kg) and methanol (510 L). Separately, a mixture of 10% palladium on carbon (0.13 Kg) in water (650 mL) is prepared and then is added to the above mass at 25-35° C. A hydrogen gas pressure of about 0.5-1 kg/cm² is applied, the mixture is heated to 50-54° C. at 2.5-3.5 kg/cm² pressure, and then the mixture is stirred at the same temperature and pressure for about 3 hours for completion of the reaction, as verified using TLC. An additional mixture of 10% palladium on carbon (0.04 Kg) in water (200 mL) is added and the mass is further stirred at 50-54° C. under hydrogen pressure for 2 hours. The mixture is cooled to about 45-50° C., filtered, and the bed is washed with methanol (17 L). Concentrated hydrochloric acid (42.5 L) is added to the filtrate at 25-30° C. over 20 minutes, and the mixture is stirred at 25-30° C. for 3-4 hours. The formed solid is collected by filtration, washed with methanol (9 L then 17 L), and dried under vacuum to afford the title compound in about 66% yield.

### EXAMPLE 60

### Preparation of Nebivolol Hydrochloride Having Small Particle Sizes

Nebivolol hydrochloride (10 g) is charged into a round bottom flask containing methanol (400 mL), and the mixture is heated to reflux with stirring to obtain a solution. The solution is cooled to about 50° C. and poured into precooled methyl t-butyl ether (800 mL), maintained at about 5-10° C., over about 15 minutes. After complete addition, the mixture is stirred at 5-10° C. for about 30 minutes. The mixture is heated to about 35° C. and maintained for 30 minutes, and then is cooled to about 15° C. over 45-60 minutes. The mixture is maintained at that temperature for an additional 1 hour and the solid is collected by filtration, washed methyl t-butyl ether (20 mL), and dried under vacuum at 50° C.

The obtained solid (8.5 g) has a particle size distribution with D₉₀=9.35 μm, D₁₀=1.43 μm, D₅₀=3.52 μm.

1. A process for preparing a compound of Formula I, where HX is an acid, comprising: 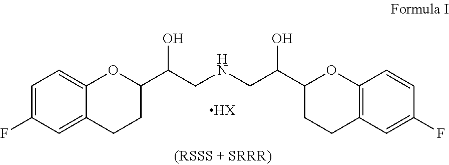
a) reacting the compound of Formula VIA or VIB with a nitrogen nucleophile, optionally in the presence of a solvent, to afford a compound of Formula VA or VB, wherein P is a protecting group residue from the nitrogen nucleophile; 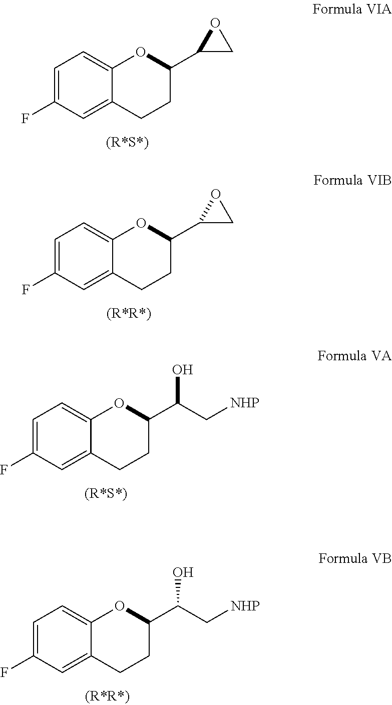
reacting a compound of Formula VIA or VIB with a compound of Formula VB or VA, optionally in the presence of a solvent, to give a compound of Formula IV; 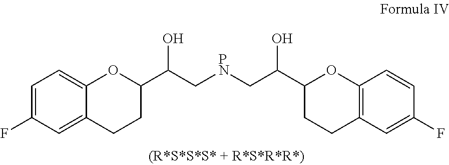
b) purifying the compound of Formula IV, by precipitating or crystallizing a compound of Formula IIIA having high isomeric purity; 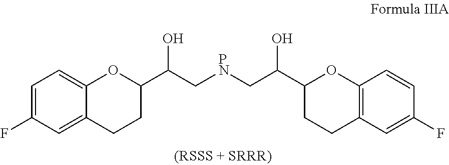
c) deprotecting the compound of Formula IIIA from step c), to give the compound of Formula IIA; and 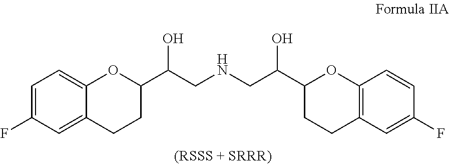
d) reacting the compound of Formula IIA with an acid HX to give a compound of Formula I.

2. The process of claim 1, wherein a nitrogen nucleophile is benzylamine. and P is benzyl.

3. The process of claim 1, wherein in step b) a solvent is an alcohol.

4. The process of claim 1, wherein in step c) purifying is accomplished from solutions with alcohol or aqueous alcohol solvents.

5. The process of claim 1, wherein in step c) purifying is accomplished by slurrying in an alcohol or aqueous alcohol.

6. The process of claim 4, wherein during crystallization the solution is cooled in a step-wise manner.

7. The process of claim 1, wherein in step d) deprotection is accomplished using hydrogen gas in the presence of less than 3% of a hydrogenation catalyst, based on the weight of a compound of Formula IIIA.

8. The process of claim 1, wherein in step d) deprotection is accomplished using hydrogen gas in the presence of less than 2% of a hydrogenation catalyst, based on the weight of a compound of Formula IIIA.

9. The process of claim 1, wherein in step e) an acid is hydrochloric acid, methanesulfonic acid, sulfuric acid, or acetic acid.

10. A process for preparing the compound of Formula IIIA, comprising:
a) reacting a compound of Formula VIA with a compound of Formula VB, or reacting a compound of Formula VIB with a compound of Formula VA, where P is a protecting group, optionally in the presence of a solvent, to afford a compound of Formula IV; and 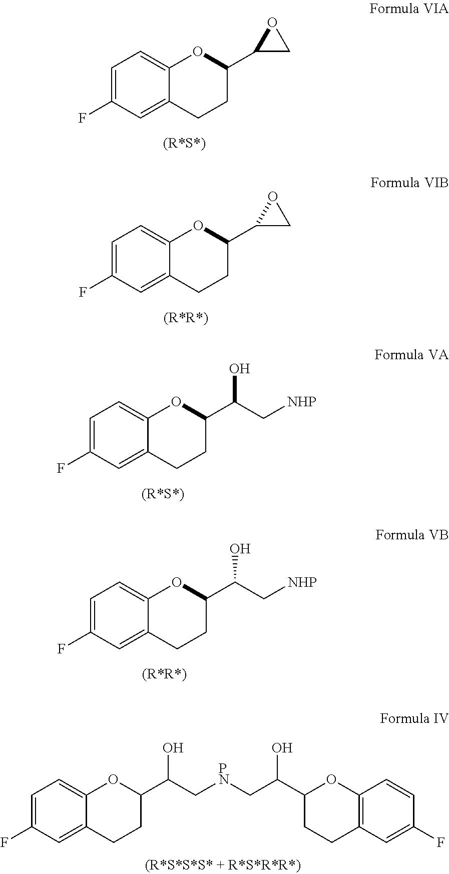
b) from a solution of a compound of Formula IV, precipitating or crystallizing a compound of Formula IIIA with substantial diastereomeric purity.

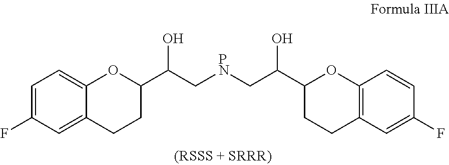

11. The process of claim 10, wherein diastereomeric purity of a compound of Formula IIIA is at least 98%.

12. A process for preparing a compound of Formula IIIA, comprising precipitating or crystallizing a compound of Formula IV from an organic or aqueous solution.

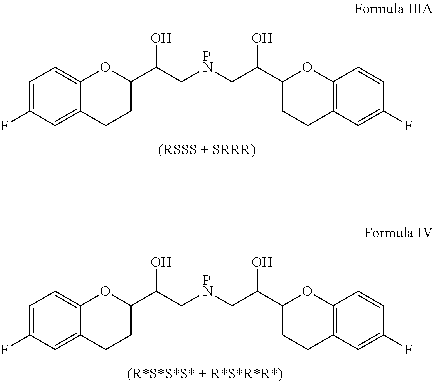

13. The process of claim 10, wherein a solution comprises an alcohol solvent.

14. A process for preparing compounds of Formulas VIA and VIB, comprising: 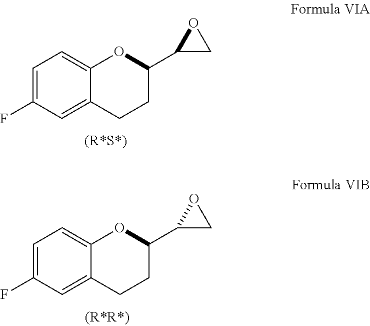
a) reacting 6-fluorochroman-carboxylic acid with a reducing agent, in a solvent, to give 6-fluorochromamethanol;
b) treating 6-fluorochromamethanol with a radical initiator, in the presence of a halogen and a base, to give 6-fluorochromancarboxaldehyde;
c) reacting 6-fluorochromancarboxaldehyde with trimethylsulfoxonium iodide, in the presence of a base and a solvent, to give a compound of Formula VI; and 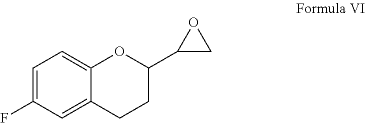
d) subjecting a compound of Formula VI to chromatographic purification, to give the compounds of Formulas VIA and VIB.

15. The process of claim 14, wherein in b) a radical initiator is TEMPO, a halogen is iodine, and a base is a bicarbonate.

16. A process for preparing particles of nebivolol hydrochloride, comprising:
a) providing a solution of nebivolol hydrochloride in a solvent;
b) adding the solution to an anti-solvent for nebivolol hydrochloride; and
c) recovering solid nebivolol hydrochloride.

17. The process of claim 16, wherein a solvent is an alcohol.

18. The process of claim 16, wherein an anti-solvent is an ether, cyclic ether, or ester.

19. Nebivolol hydrochloride prepared by the process of claim 16 and having a particle size distribution where D₉₀ is less than 40 μm.
